(19) United States
(12) Patent Application Publication
(10) Pub. No.: US 2024/0400114 A1
(43) Pub. Date: Dec. 5, 2024

(54) BRAINWAVE ANALYSIS FOR TRANSFER OF CONTROL IN SEMI-AUTONOMOUS VEHICLES

(71) Applicant: Toyota Motor Engineering & Manufacturing North America, Inc., Plano, TX (US)

(72) Inventor: Raymond P. Viviano, Hazel Park, MI (US)

(73) Assignees: Toyota Motor Engineering & Manufacturing North America, Inc., Plano, TX (US); Toyota Jidosha Kabushiki Kaisha, Aichi-ken (JP)

(21) Appl. No.: 18/327,635

(22) Filed: Jun. 1, 2023

(51) Int. Cl. B60W 60/00; A61B 5/16; A61B 5/18; A61B 5/374; B60W 10/18; B60W 10/20; B60W 50/14

(52) U.S. Cl. CPC B60W 60/0054; A61B 5/163; A61B 5/18; A61B 5/374; B60W 10/18; B60W 10/20; B60W 50/14; B60W 2050/143; B60W 2050/146; B60W 2540/12; B60W 2540/18; B60W 2540/225

(57) ABSTRACT

A vehicle control system is provided for use with a semi-autonomous vehicle. The vehicle control system includes: a hazard-detection system configured to output a hazard-detection signal based on a detected hazard; an eye-tracking system configured to output an eye-tracking signal based on where the driver is looking; a brain-function system configured to output a brain-function signal based on a detected parameter; and a driving-control system configured to determine whether the driver comprehends the hazard based on the hazard-detection signal, the eye-tracking signal and the brain-function signal, and operate in a driver-controlled state so as to enable the driver to control at least one of steering of the semi-autonomous vehicle, breaking of the semi-autonomous vehicle, and a combination thereof, and a driving-control-system-controlled state so as to enable the driving-control system to control the at least one of steering of the semi-autonomous vehicle, breaking of the semi-autonomous vehicle, and a combination thereof.

### TECHNICAL FIELD

One or more embodiments relate generally semi-autonomous vehicles, and systems and method of operating semi-autonomous vehicles.

### BACKGROUND

The Society of Automotive Engineers (SAE) has defined five levels of autonomous driving, based on the degree of human intervention and control required. Level 1 is driver assistance, wherein the vehicle has some automated functions, such as cruise control or lane departure warning, but the driver is still responsible for most aspects of driving. Level 2 is partial automation, wherein the vehicle has more advanced driver assistance systems, such as automated braking and lane centering, but the driver must still be attentive and ready to take control at any time. Level 3 is conditional automation, wherein the vehicle can handle most driving tasks under certain conditions, such as on a highway, but the driver must still be able to take control if needed. The driver can be less attentive but must still be ready to intervene. Level 4 is high automation, wherein the vehicle can perform all driving tasks under certain conditions, and the driver is not required to intervene. However, the vehicle may still require human input in some situations, such as extreme weather. Level 5 is full automation, wherein the vehicle can perform all driving tasks in any conditions, and no human input is required. Level 5 autonomy does not exist yet, but it is the ultimate goal of autonomous vehicle development.

For purposes of this disclosure, levels 2-4 may be generally described as semi-autonomous driving, as there is some driver control needed at least at some times.

An issue with semi-autonomous driving systems is drawn to hazard recognition and avoidance. Some conventional semi-autonomous driving systems are able to recognize driving hazards, such as: impending collisions with other vehicles; impending collisions with inanimate objects, such as buildings, trees, guard rails, curbs, trash cans, etc.; and impending collisions with people or animals. Further, some of these semi-autonomous driving systems may attempt to autonomously control steering and breaking to avoid these hazards. However, in some situations, wherein the driver also tries to control steering and breaking to avoid these hazards, some control conflicts may arise that ultimately result in neither the driver nor the semi-autonomous driving system being able to avoid the hazard.

What is needed is a semi-autonomous driving system that is able to recognize a hazard and autonomously control breaking and/or steering to avoid the hazard, only when the driver is not controlling breaking and/or steering to avoid the hazard.

### SUMMARY

An aspect of the present disclosure is drawn to a vehicle control system for use with a semi-autonomous vehicle, wherein the vehicle control system includes: a hazard-detection system including: a hazard-detection memory having hazard-detection instructions stored therein; and a hazard-detection processor configured to execute the hazard-detection instructions to cause the hazard-detection system to: detect a hazard; and output a hazard-detection signal based on the detected hazard; an eye-tracking system including: an eye-tracking memory having eye-tracking instructions stored therein; and an eye-tracking processor configured to execute the eye-tracking instructions to cause the eye-tracking system to: detect where a driver of the semi-autonomous vehicle is looking; and output an eye-tracking signal based on where the driver is looking; a brain-function system including: a brain-function memory having brain-function instructions stored therein; and a brain-function processor configured to execute the brain-function instructions to cause the brain-function system to: detect a parameter of a brain of the driver; and output a brain-function signal based on the detected parameter; and a driving-control system including: a driving-control memory having driving-control instructions stored therein; and a driving-control processor configured to execute the driving-control instructions to cause the driving-control system to: determine whether the driver comprehends the hazard based on the hazard-detection signal, the eye-tracking signal and the brain-function signal; and operate in: a driver-controlled state so as to enable the driver to control at least one of steering of the semi-autonomous vehicle, breaking of the semi-autonomous vehicle, and a combination thereof; and a driving-control-system-controlled state so as to enable the driving-control system to control the at least one of steering of the semi-autonomous vehicle, breaking of the semi-autonomous vehicle, and a combination thereof.

In some embodiments of this aspect, the vehicle control system further includes a warning system configured to provide a warning related to the driving-control system controlling the at least one of steering of the semi-autonomous vehicle, breaking of the semi-autonomous vehicle, and a combination thereof when the driving-control system is operating in the driving-control system state. In some of these embodiments, the warning system comprises a display configured to display an image related to the driving-control system controlling the at least one of steering of the semi-autonomous vehicle, breaking of the semi-autonomous vehicle, and a combination thereof when the driving-control system is operating in the driving-control system state. In some of these embodiments, the display comprises a head up display.

In some embodiments of this aspect that further includes a warning system configured to provide a warning related to the driving-control system controlling the at least one of steering of the semi-autonomous vehicle, breaking of the semi-autonomous vehicle, and a combination thereof when the driving-control system is operating in the driving-control system state, the warning system includes a speaker configured to output an audible warning related to the driving-control system controlling the at least one of steering of the semi-autonomous vehicle, breaking of the semi-autonomous vehicle, and a combination thereof when the driving-control system is operating in the driving-control system state.

In some embodiments of this aspect, the driving-control processor is further configured to execute the driving-control instructions to additionally cause the driving-control system to: determine whether the semi-autonomous vehicle has cleared the hazard; and operate in the driver-controlled state if the semi-autonomous vehicle has cleared the hazard and when previously operating in the driving-control-system-controlled state.

In some embodiments of this aspect, the brain-function processor is further configured to execute the brain-function instructions to cause the brain-function system to detect a gamma power in a visual cortex part of the brain as the parameter of a brain of the driver.

An aspect of the present disclosure is drawn to a method of operating an semi-autonomous vehicle, wherein the method includes: detecting, via hazard-detection processor configured to execute hazard-detection instructions stored in a hazard-detection memory, a hazard; outputting, via the hazard-detection processor, a hazard-detection signal based on the detected hazard; detecting, via an eye-tracking processor configured to execute eye-tracking instructions stored in an eye-tracking memory, where a driver of the semi-autonomous vehicle is looking; outputting, via the eye-tracking processor, an eye-tracking signal based on where the driver is looking; detecting, via a brain-function processor configured to execute the brain-function instructions stored in a brain-function memory, a parameter of a brain of the driver; outputting, via the brain-function processor, a brain-function signal based on the detected parameter; determining, via a driving-control processor configured to execute the driving-control instructions stored in a driving-control memory, whether the driver comprehends the hazard based on the hazard-detection signal, the eye-tracking signal and the brain-function signal; and operating the driving-control processor in: a driver-controlled state so as to enable the driver to control at least one of steering of the semi-autonomous vehicle, breaking of the semi-autonomous vehicle, and a combination thereof; and a driving-control-system-controlled state so as to enable the driving-control system to control the at least one of steering of the semi-autonomous vehicle, breaking of the semi-autonomous vehicle, and a combination thereof.

In some embodiments of this aspect, the method further includes providing, via a warning system, a warning related to the driving-control system controlling the at least one of steering of the semi-autonomous vehicle, breaking of the semi-autonomous vehicle, and a combination thereof when the driving-control system is operating in the driving-control system state. In some of these embodiments, the comprising a warning comprises displaying, via a display, an image related to the driving-control system controlling the at least one of steering of the semi-autonomous vehicle, breaking of the semi-autonomous vehicle, and a combination thereof when the driving-control system is operating in the driving-control system state. In some of these embodiments, the displaying the image comprises displaying the image via a head up display.

In some embodiments of this aspect, wherein the method further includes providing, via a warning system, a warning related to the driving-control system controlling the at least one of steering of the semi-autonomous vehicle, breaking of the semi-autonomous vehicle, and a combination thereof when the driving-control system is operating in the driving-control system state, the comprising a warning comprises outputting, via a speaker, an audible warning related to the driving-control system controlling the at least one of steering of the semi-autonomous vehicle, breaking of the semi-autonomous vehicle, and a combination thereof when the driving-control system is operating in the driving-control system state.

In some embodiments of this aspect, the method further includes: determining, via the driving-control processor, whether the semi-autonomous vehicle has cleared the hazard; and operating, via the driving-control processor, in the driver controlled state if the semi-autonomous vehicle has cleared the hazard and when previously operating in the driving-control-system-controlled state.

In some embodiments of this aspect, the method further includes detecting, via the brain-function processor, a gamma power in a visual cortex part of the brain as the parameter of the brain of the driver.

An aspect of the present disclosure is drawn to a non-transitory, computer-readable media having computer-readable instructions stored thereon, the computer-readable instructions being capable of being read by a vehicle control system for use with an semi-autonomous vehicle, wherein the computer-readable instructions are capable of instructing the vehicle control system to perform the method including: detecting, via hazard-detection processor configured to execute hazard-detection instructions stored in a hazard-detection memory, a hazard; outputting, via the hazard-detection processor, a hazard-detection signal based on the detected hazard; detecting, via an eye-tracking processor configured to execute eye-tracking instructions stored in an eye-tracking memory, where a driver of the semi-autonomous vehicle is looking; outputting, via the eye-tracking processor, an eye-tracking signal based on where the driver is looking; detecting, via a brain-function processor configured to execute the brain-function instructions stored in a brain-function memory, a parameter of a brain of the driver; outputting, via the brain-function processor, a brain-function signal based on the detected parameter; determining, via a driving-control processor configured to execute the driving-control instructions stored in a driving-control memory, whether the driver comprehends the hazard based on the hazard-detection signal, the eye-tracking signal and the brain-function signal; and operating the driving-control processor in: a driver-controlled state so as to enable the driver to control at least one of steering of the semi-autonomous vehicle, breaking of the semi-autonomous vehicle, and a combination thereof; and a driving-control-system-controlled state so as to enable the driving-control system to control the at least one of steering of the semi-autonomous vehicle, breaking of the semi-autonomous vehicle, and a combination thereof.

In some of these embodiments of this aspect, the computer-readable instructions are capable of instructing the vehicle control system to perform the method further including providing, via a warning system, a warning related to the driving-control system controlling the at least one of steering of the semi-autonomous vehicle, breaking of the semi-autonomous vehicle, and a combination thereof when the driving-control system is operating in the driving-control system state. In some of these embodiments, the computer-readable instructions are capable of instructing the vehicle control system to perform the method wherein the comprising a warning comprises displaying, via a display, an image related to the driving-control system controlling the at least one of steering of the semi-autonomous vehicle, breaking of the semi-autonomous vehicle, and a combination thereof when the driving-control system is operating in the driving-control system state. In some of these embodiments, the computer-readable instructions are capable of instructing the vehicle control system to perform the method wherein the displaying the image comprises displaying the image via a head up display.

In some embodiments of this aspect, wherein the computer-readable instructions are capable of instructing the vehicle control system to perform the method further including providing, via a warning system, a warning related to the driving-control system controlling the at least one of steering of the semi-autonomous vehicle, breaking of the semi-autonomous vehicle, and a combination thereof when the driving-control system is operating in the driving-control system state, the computer-readable instructions are further capable of instructing the vehicle control system to perform the method wherein the comprising a warning comprises outputting, via a speaker, an audible warning related to the driving-control system controlling the at least one of steering of the semi-autonomous vehicle, breaking of the semi-autonomous vehicle, and a combination thereof when the driving-control system is operating in the driving-control system state.

In some embodiments of this aspect, the computer-readable instructions are capable of instructing the vehicle control system to perform the method further includes: determining, via the driving-control processor, whether the semi-autonomous vehicle has cleared the hazard; and operating, via the driving-control processor, in the driver controlled state if the semi-autonomous vehicle has cleared the hazard and when previously operating in the driving-control-system-controlled state.

In some embodiments of this aspect, the computer-readable instructions are capable of instructing the vehicle control system to perform the method further including detecting, via the brain-function processor, a gamma power in a visual cortex part of the brain as the parameter of the brain of the driver.

An aspect of the present disclosure is drawn to a system including: a semi-autonomous vehicle configured to semi-autonomously drive; and a vehicle control system incorporated into the semi-autonomous vehicle and including: a hazard-detection system including: a hazard-detection memory having hazard-detection instructions stored therein; and a hazard-detection processor configured to execute the hazard-detection instructions to cause the hazard-detection system to: detect a hazard; and output a hazard-detection signal based on the detected hazard; an eye-tracking system including: an eye-tracking memory having eye-tracking instructions stored therein; and an eye-tracking processor configured to execute the eye-tracking instructions to cause the eye-tracking system to: detect where a driver of the semi-autonomous vehicle is looking; and output an eye-tracking signal based on where the driver is looking; a brain-function system including: brain-function memory having brain-function instructions stored therein; and a brain-function processor configured to execute the brain-function instructions to cause the brain-function system to: detect a parameter of a brain of the driver; and output a brain-function signal based on the detected parameter; and a driving-control system including: a driving-control memory having driving-control instructions stored therein; and a driving-control processor configured to execute the driving-control instructions to cause the driving-control system to: determine whether the driver comprehends the hazard based on the hazard-detection signal, the eye-tracking signal and the brain-function signal; and operate in: a driver-controlled state so as to enable the driver to control at least one of steering of the semi-autonomous vehicle, breaking of the semi-autonomous vehicle, and a combination thereof; and a driving-control-system-controlled state so as to enable the driving-control system to control the at least one of steering of the semi-autonomous vehicle, breaking of the semi-autonomous vehicle, and a combination thereof.

In some of these embodiments, the vehicle control system further includes a warning system configured to provide a warning related to the driving-control system controlling the at least one of steering of the semi-autonomous vehicle, breaking of the semi-autonomous vehicle, and a combination thereof when the driving-control system is operating in the driving-control system state. In some of these embodiments, the warning system includes a display configured to display an image related to the driving-control system controlling the at least one of steering of the semi-autonomous vehicle, breaking of the semi-autonomous vehicle, and a combination thereof when the driving-control system is operating in the driving-control system state. In some of these embodiments, the display comprises a head up display.

In some of these embodiments, wherein the vehicle control system further includes a warning system configured to provide a warning related to the driving-control system controlling the at least one of steering of the semi-autonomous vehicle, breaking of the semi-autonomous vehicle, and a combination thereof when the driving-control system is operating in the driving-control system state, the warning system includes a speaker configured to output an audible warning related to the driving-control system controlling the at least one of steering of the semi-autonomous vehicle, breaking of the semi-autonomous vehicle, and a combination thereof when the driving-control system is operating in the driving-control system state.

In some of these embodiments, the driving-control processor is further configured to execute the driving-control instructions to additionally cause the driving-control system to: determine whether the semi-autonomous vehicle has cleared the hazard; and operate in the driver-controlled state if the semi-autonomous vehicle has cleared the hazard and when previously operating in the driving-control-system-controlled state.

In some of these embodiments, the brain-function processor is further configured to execute the brain-function instructions to cause the brain-function system to detect a gamma power in a visual cortex part of the brain as the parameter of a brain of the driver.

### BRIEF SUMMARY OF THE DRAWINGS

The accompanying drawings, which are incorporated in and form a part of the specification, illustrate and explain example embodiments. In the drawings:

FIG. 1 illustrates an example process to be executed by a processor for controlling a semi-autonomous vehicle in accordance with aspects of the present disclosure;

FIG. 2A illustrates an autonomous hazard detection and avoidance system (AHDAS) for controlling a semi-autonomous vehicle at a time t₀ in accordance with aspects of the present disclosure;

FIG. 2B illustrates the AHDAS of FIG. 2A at a time t₁ in accordance with aspects of the present disclosure;

FIG. 2C illustrates the AHDAS of FIG. 2A e at a time t₂ in accordance with aspects of the present disclosure;

FIG. 2D illustrates the AHDAS of FIG. 2A at a time t₃ in accordance with aspects of the present disclosure;

FIG. 2E illustrates the AHDAS of FIG. 2A at a time t₄ in accordance with aspects of the present disclosure;

FIG. 2F illustrates the AHDAS of FIG. 2A at a time t₅ in accordance with aspects of the present disclosure;

FIG. 2G illustrates the AHDAS of FIG. 2A at a time t₆ in accordance with aspects of the present disclosure;

FIG. 2H illustrates the AHDAS of FIG. 2A at a time t₇ in accordance with aspects of the present disclosure;

FIG. 3 illustrates a more detailed view of the hazard-detection system of the system of FIGS. 2A-E;

FIG. 4 illustrates a more detailed view of the eye-tracking system of the system of FIGS. 2A-E;

FIG. 5 illustrates a more detailed view of the EEG-analyzing system of the system of FIGS. 2A-E;

FIG. 6 illustrates multiple views of a human brain;

FIG. 7 illustrates sustained gamma power graphs of a human brain;

FIG. 8 illustrates convolved gamma power graphs of a human brain;

FIG. 9 illustrates a dorsal attention network, a ventral attention network, and a visual cortex of a human brain;

FIG. 10 illustrate a graph of voltage displacements of an EEG signal related to novel stimulus processing;

FIG. 11 illustrates signal phase of a brain network; and

FIG. 12 illustrates a more detailed view of the driving-control system of the system of FIGS. 2A-E.

### DETAILED DESCRIPTION

A semi-autonomous vehicle in accordance with aspects of the present disclosure includes an autonomous hazard detection and avoidance system (AHDAS) that is able to recognize a hazard and autonomously control breaking and/or steering to avoid the hazard, only when the driver is not controlling breaking and/or steering to avoid the hazard.

A semi-autonomous vehicle having an AHDAS in accordance with aspects of the present disclosure combines electroencephalogram (EEG) neural signals, eye-tracking gaze direction data, and driver behavior monitoring to determine if a driver is responding appropriately to a road hazard. If the driver responds to the hazard in a safe manner, the semi-autonomous vehicle does not assume control of driving functions; however, the semi-autonomous vehicle does assume control if the driver is either inattentive or makes poor decisions regarding the hazard.

In a semi-autonomous vehicle having AHDAS in accordance with aspects of the present disclosure, the AHDAS monitors road conditions and potential hazards through arrays of external sensors (e.g., LIDAR, RADAR, thermal imaging, acoustic, etc.). The AHDAS simultaneously monitors driver eye-gaze direction with an eye-tracking camera and monitors attention by processing EEG signals. When the AHDAS detects a significant road hazard, it displays a warning on a head-up display (HUD) and plays an audio alert to direct driver attention towards the hazard. The AHDAS then evaluates if the driver has directed their gaze to the hazard and comprehended the danger through EEG evaluation. The AHDAS also monitors acceleration/deceleration, braking, and steering behaviors. If the AHDAS determines that the driver has not adequately comprehended the hazard and the driver does not take corrective action, the AHDAS conditionally transfers control to an autonomous driving state until the semi-autonomous vehicle has passed the hazard.

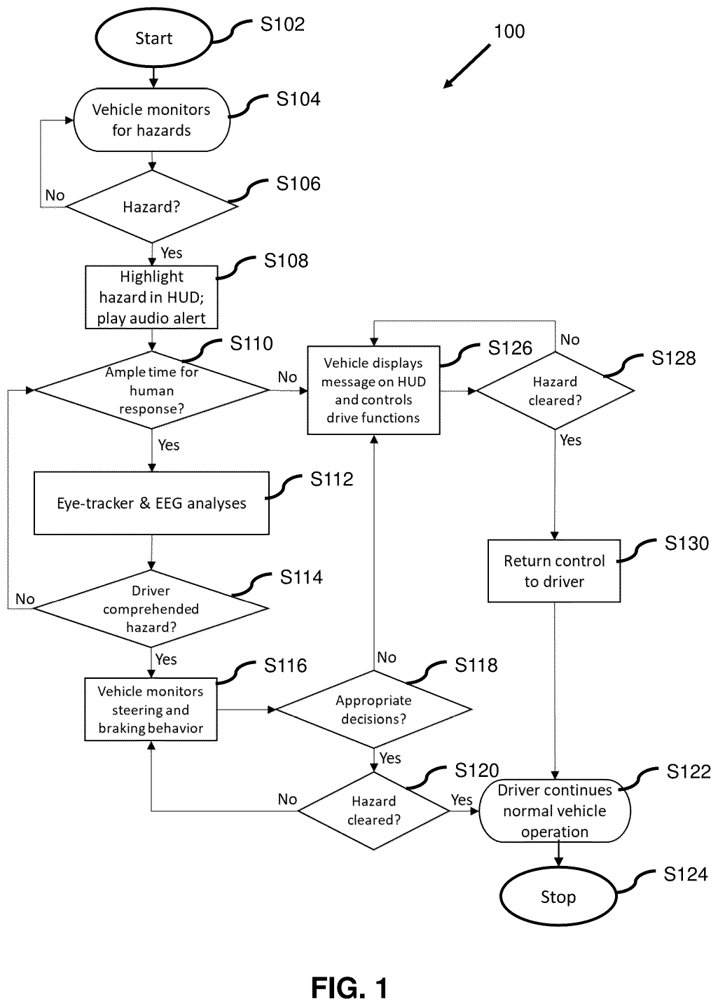

An example AHDAS and a process to be executed by a processor in accordance with aspects of the present disclosure will now be described in greater detail with reference to FIGS. 1-12.

FIG. 1 illustrates an example process 100 to be executed by a processor for controlling a semi-autonomous vehicle in accordance with aspects of the present disclosure.

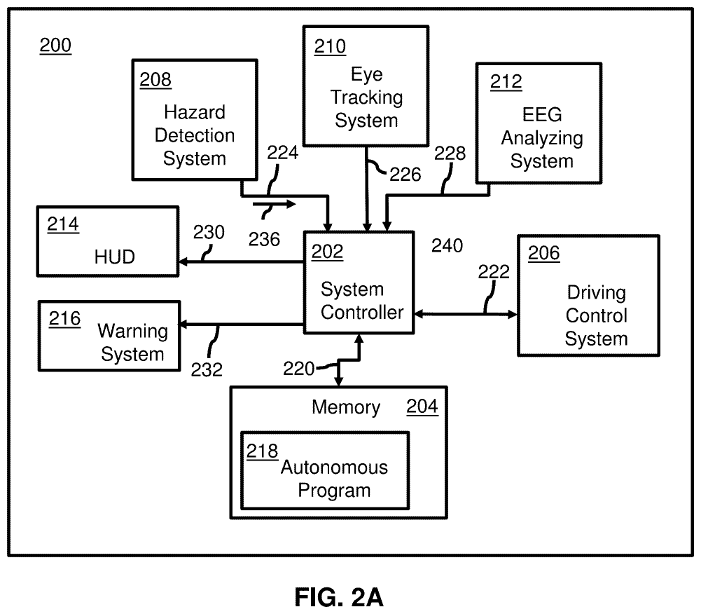

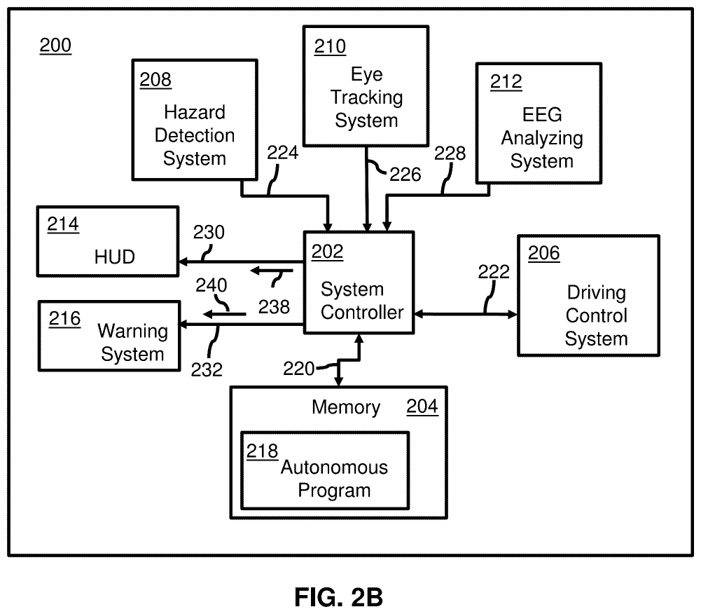

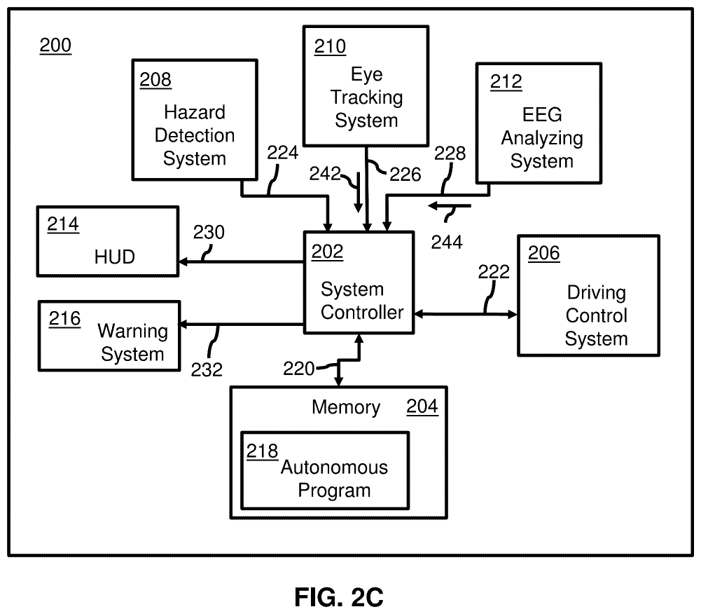

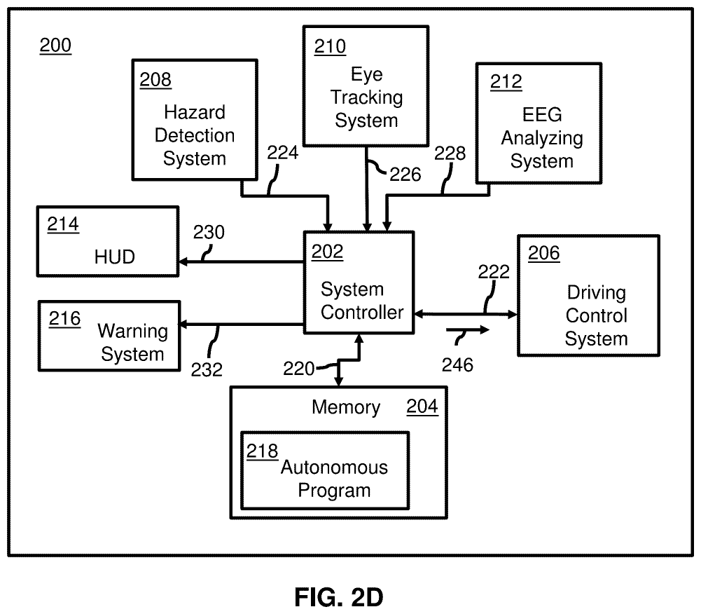

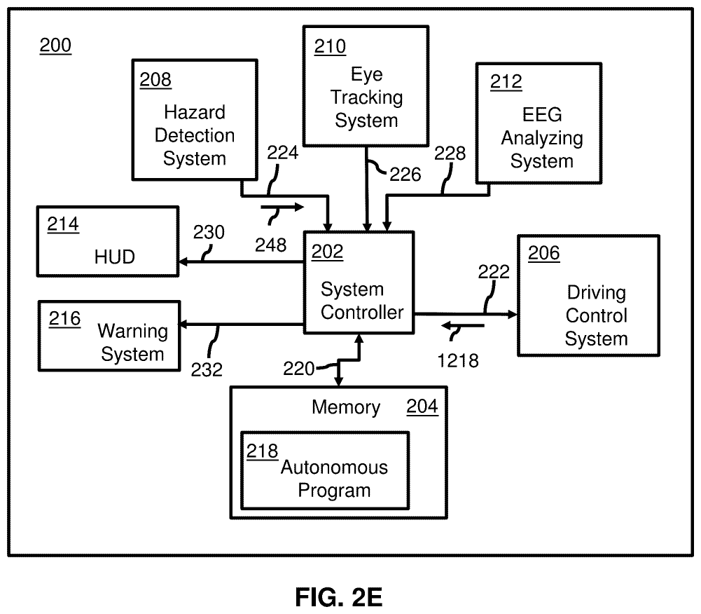

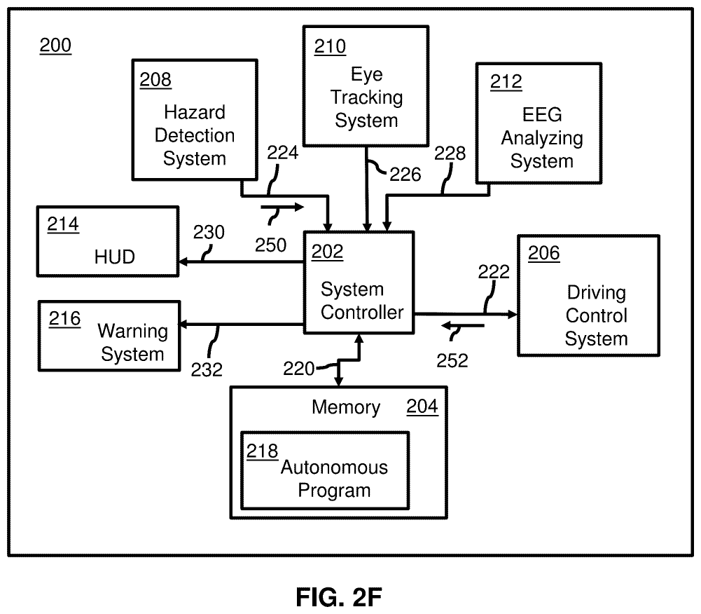

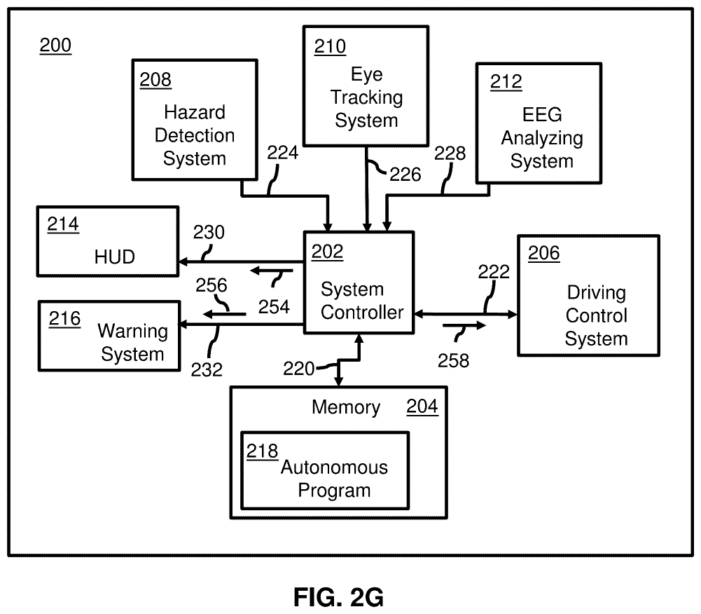

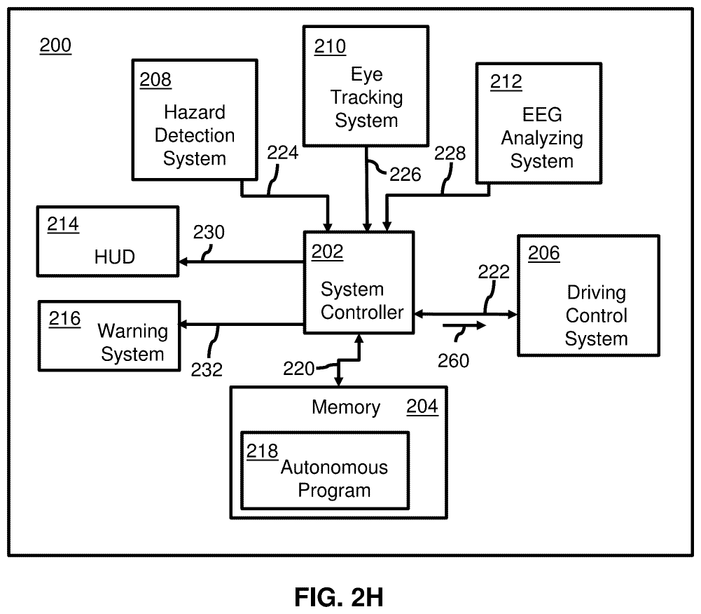

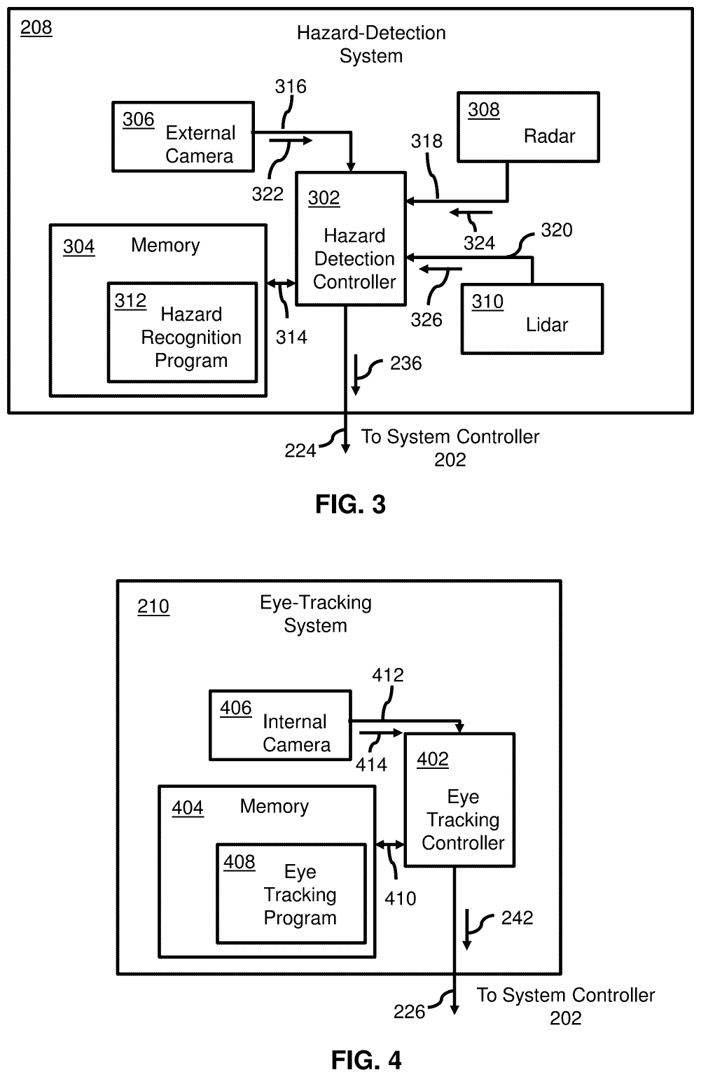

As shown in the figure, process 100 starts (S102) and the semi-autonomous vehicle monitors for hazards (S104). This will be described in greater detail with reference to FIGS. 2A and 3.

FIG. 2A illustrates an AHDAS 200 for controlling a semi-autonomous vehicle at a time t₀ in accordance with aspects of the present disclosure.

As shown in the figure, AHDAS 200 includes an AHDAS controller 202, a AHDAS memory 204, a driving-control system 206, a hazard-detection system 208, an eye-tracking system 210, an electroencephalogram (EEG) analyzing system 212, a head up display (HUD) 214, and a warning system 216. AHDAS memory 204 has stored therein data and instructions including an autonomous program 218 to be executed by AHDAS controller 202. AHDAS controller 202 is configured to execute instructions in autonomous program 218 of AHDAS memory 204.

AHDAS controller 202 may be any device or system that is configured to control general operations of AHDAS 200 and includes, but is not limited to, a central processing unit (CPU), a hardware microprocessor, a single core processor, a multi-core processor, a field programmable gate array (FPGA), a microcontroller, an application specific integrated circuit (ASIC), a digital signal processor (DSP), or other similar processing device capable of executing any type of instructions, algorithms, or software for controlling the operation and functions of AHDAS 200.

AHDAS memory 204 may be any device or system capable of storing data and instructions used by AHDAS 200 and includes, but is not limited to, random-access memory (RAM), dynamic random-access memory (DRAM), a hard drive, a solid-state drive, read-only memory (ROM), erasable programmable read-only memory (EPROM), electrically erasable programmable read-only memory (EEPROM), flash memory, embedded memory blocks in an FPGA, or any other various layers of memory hierarchy.

In this example, AHDAS controller 202, AHDAS memory 204, driving-control system 206, hazard-detection system 208, eye-tracking system 210, EEG-analyzing system 212, HUD 214, and warning system 216 are illustrated as individual devices of AHDAS 200. However, in some embodiments, at least two of AHDAS controller 202, AHDAS memory 204, driving-control system 206, hazard-detection system 208, eye-tracking system 210, EEG-analyzing system 212, HUD 214, and warning system 216 may be combined as a unitary device. Further, in some embodiments, at least one of AHDAS controller 202, AHDAS memory 204, driving-control system 206, hazard-detection system 208, eye-tracking system 210, EEG-analyzing system 212, HUD 214, and warning system 216 may be implemented as a computer having non-transitory computer-readable media for carrying or having computer-executable instructions or data structures stored thereon. Such non-transitory computer-readable recording medium refers to any computer program product, apparatus or device, such as a magnetic disk, optical disk, solid-state storage device, memory, programmable logic devices (PLDs), DRAM, RAM, ROM, EEPROM, CD-ROM or other optical disk storage, magnetic disk storage or other magnetic storage devices, or any other medium that can be used to carry or store desired computer-readable program code in the form of instructions or data structures and that can be accessed by a general-purpose or special-purpose computer, or a general-purpose or special-purpose processor. Disk or disc, as used herein, includes compact disc (CD), laser disc, optical disc, digital versatile disc (DVD), floppy disk and Blu-ray disc. Combinations of the above are also included within the scope of computer-readable media. For information transferred or provided over a network or another communications connection (either hardwired, wireless, or a combination of hardwired or wireless) to a computer, the computer may properly view the connection as a computer-readable medium. Thus, any such connection may be properly termed a computer-readable medium. Combinations of the above should also be included within the scope of computer-readable media.

Example tangible computer-readable media may be coupled to AHDAS controller 202 such that the processor may read information from and write information to the tangible computer-readable media. In the alternative, the tangible computer-readable media may be integral to AHDAS controller 202. AHDAS controller 202 and the tangible computer-readable media may reside in an integrated circuit (IC), an ASIC, or large-scale integrated circuit (LSI), system LSI, super LSI, or ultra LSI components that perform a part or all of the functions described herein. In the alternative, AHDAS controller 202 and the tangible computer-readable media may reside as discrete components.

Example tangible computer-readable media may be also coupled to systems, non-limiting examples of which include a computer system/server, which is operational with numerous other general purpose or special purpose computing system environments or configurations. Examples of well-known computing systems, environments, and/or configurations that may be suitable for use with computer system/server include, but are not limited to, personal computer systems, server computer systems, thin clients, thick clients, handheld or laptop devices, multiprocessor systems, microprocessor-based systems, set-top boxes, programmable consumer electronics, network PCs, minicomputer systems, mainframe computer systems, and distributed cloud computing environments that include any of the above systems or devices, and the like.

Such a computer system/server may be described in the general context of computer system-executable instructions, such as program modules, being executed by a computer system. Generally, program modules may include routines, programs, objects, components, logic, data structures, and so on that perform particular tasks or implement particular abstract data types. Further, such a computer system/server may be practiced in distributed cloud computing environments where tasks are performed by remote processing devices that are linked through a communications network. In a distributed cloud computing environment, program modules may be located in both local and remote computer system storage media including memory storage devices.

Autonomous program 218 controls the operations of AHDAS 200. Autonomous program 218, having a set (at least one) of program modules, may be stored in AHDAS memory 204 by way of example, and not limitation, as well as an operating system, one or more application programs, other program modules, and program data. Each of the operating system, one or more application programs, other program modules, and program data or some combination thereof, may include an implementation of a networking environment. The program modules generally carry out the functions and/or methodologies of various embodiments of the application as described herein.

AHDAS controller 202 is configured: to communicate with AHDAS memory 204 via a communication channel 220; to communicate with driving-control system 206 via a communication channel 222; to communicate with hazard-detection system 208 via a communication channel 224; to communicate with eye-tracking system 210 via a communication channel 226; to communicate with EEG-analyzing system 212 via a communication channel 228; to communicate with HUD 214 via a communication channel 230; and to communicate with warning system 216 via a communication channel 232.

Driving-control system 206 may be any device or system that is configured to monitor and control the steering and braking of the semi-autonomous vehicle.

Hazard-detection system 208 may be any device or system that is configured to identify potential dangers on the road. Hazard-detection system 208 may include various sensors, such as cameras, radar, and lidar, to detect and track objects and obstacles in the semi-autonomous vehicle's path. Hazard-detection system 208 is configured to continuously analyze the data from the sensors and make real-time identifications of hazards.

Eye-tracking system 210 may be any device or system that is configured to track an eye of the driver to determine where the driving is looking.

EEG-analyzing system 212 may be any device or system that is configured to detect a parameter of the brain of the driver to determine whether the attention of the driver is directed externally (to the visual environment) rather than internally (daydreaming), and whether the driver recognizes a hazard.

HUD 214 may be any device or system that is configured to project information onto the windshield of the semi-autonomous vehicle, allowing the driver to view important information without having to look down at the dashboard or take their eyes off the road.

HUD 214 may include a projector located near the dashboard of the semi-autonomous vehicle that projects the information onto a small portion of the windshield, usually near the driver's line of sight. A reflective material may be included on the windshield to reflect the projected image back to the driver, allowing them to see the information without having to look away from the road.

Warning system 216 may be any device or system that is configured to: provide a warning of a hazard; and provide a warning related to driving-control system 206 controlling the at least one of steering of the semi-autonomous vehicle, breaking of the semi-autonomous vehicle, and a combination thereof when driving-control system 206 is operating in the driving-control system state. In some embodiments, warning system 216 includes a speaker configured to: an audible hazard warning related to the hazard; and output an audible autonomous control warning related to the driving-control system 206 controlling the at least one of steering of the semi-autonomous vehicle, breaking of the semi-autonomous vehicle, and a combination thereof when driving-control system 206 is operating in the driving-control system state. In some embodiments, the audible hazard warning is different from the audible autonomous control warning in one of volume, pitch, content, and combinations thereof, so that the driver may distinguish between a warning of a hazard and a warning of the autonomous control of the semi-autonomous vehicle.

In operation, hazard-detection system determines whether there is a hazard. This will be described in greater detail with reference to FIG. 3.

FIG. 3 illustrates a more detailed view of hazard-detection system 208 of AHDAS 200.

As shown in the figure, hazard-detection system 208 includes a hazard detection controller 302, a memory 304, an external camera 306, a radar 308, and a lidar 310. Memory 304 has stored therein data and instructions including a hazard-recognition program 312 to be executed by hazard detection controller 302. Hazard detection controller 302 is configured to execute instructions in hazard-recognition program 312 of memory 304.

Hazard detection controller 302 may be any device or system that is configured to control general operations of hazard-detection system 208 and includes, but is not limited to, a CPU, a hardware microprocessor, a single core processor, a multi-core processor, an FPGA, a microcontroller, an ASIC, a DSP, or other similar processing device capable of executing any type of instructions, algorithms, or software for controlling the operation and functions of hazard-detection system 208.

Memory 304 may be any device or system capable of storing data and instructions used by hazard-detection system 208 and includes, but is not limited to, RAM, DRAM, a hard drive, a solid-state drive, ROM, EPROM, EEPROM, flash memory, embedded memory blocks in an FPGA, or any other various layers of memory hierarchy.

In this example, hazard detection controller 302, memory 304, external camera 306, radar 308, and lidar 310 are illustrated as individual devices of hazard-detection system 208. However, in some embodiments, at least two of hazard detection controller 302, memory 304, external camera 306, radar 308, and lidar 310 may be combined as a unitary device. Further, in some embodiments, at least one of hazard detection controller 302, memory 304, external camera 306, radar 308, and lidar 310 may be implemented as a computer having non-transitory computer-readable media for carrying or having computer-executable instructions or data structures stored thereon.

Hazard-recognition program 312 controls the operations of hazard-detection system 208. Hazard-recognition program 312, having a set (at least one) of program modules, may be stored in memory 304 by way of example, and not limitation, as well as an operating system, one or more application programs, other program modules, and program data. Each of the operating system, one or more application programs, other program modules, and program data or some combination thereof, may include an implementation of a networking environment. The program modules generally carry out the functions and/or methodologies of various embodiments of the application as described herein.

As will be described in greater detail below, in some embodiments, hazard-recognition program 312 includes instructions, that when executed by hazard detection controller 302, cause hazard-detection system 208 to detect a hazard and output a hazard-detection signal based on the detected hazard.

Hazard detection controller 302 is configured: to communicate with memory 304 via a communication channel 314; to communicate with external camera 306 via a communication channel 316; to communicate with radar 308 via a communication channel 318; to communicate with lidar 310 via a communication channel 320; and to communicate with AHDAS controller 202 via communication channel 224.

External camera 306 may be any known type of camera, including visible, infrared (IR), ultraviolet (UV), or combination thereof, that is able to transmit image data 322, to hazard detection controller 302 via communication channel 316, of an area surrounding the semi-autonomous vehicle in real time. The image data 322 may take the form of any known image or video standard, non-limiting examples of which include JPEG, PNG, GIF, TIFF, MPR, MOV, MPEG, WMV, AVI, etc.

Radar 308 may be any known device or system that is configured to use radio waves to detect and locate objects in the semi-autonomous vehicle's path. In operation, radar 308 emits a radio wave signal, which travels through the air at the speed of light. The radio wave signal encounters an object in the semi-autonomous vehicle's path, such as another vehicle, pedestrian, or obstacle. Some of the radio wave energy is reflected back to radar 308. Radar 308 detects the reflected signal and measures the time it took for the signal to travel to the object and back. Based on the time delay, radar 308 determines the distance to the object. Further, radar 308 analyzes changes in the frequency of the reflected signal to determine the object's speed and direction of movement. Radar 308 then transmits a radar detection signal 324 to hazard detection controller 302 via communication channel 318 when an object's position, speed, and direction of movement indicate a collision course with the vehicle.

Lidar 310 may be any known device or system that is configured to use laser pulses to detect and measure objects in the semi-autonomous vehicle's environment. In operation, lidar 310 emits short pulses of laser light in various directions, for example in a 360-degree field of view. The laser light travels through the air and reflects off objects in the semi-autonomous vehicle's environment, such as other vehicles, pedestrians, and obstacles. The reflected light returns to lidar 310, where it is detected by a sensitive photodetector. Lidar 310 measures the time it took for the laser pulse to travel to the object and back. By knowing the speed of light, lidar 310 calculates the distance to the object. Lidar 310 may repeat this process many times per second, building up a 3D map of the semi-autonomous vehicle's environment. Lidar 310 then transmits a lidar detection signal 326 to hazard detection controller 302 via communication channel 320 when an object's position, speed, and direction of movement indicate a collision course with the semi-autonomous vehicle.

Returning to FIG. 1, after the semi-autonomous vehicle monitors for hazards (S104), it is determined whether a hazard exists (S106). For example, as shown in FIG. 3, hazard detection controller 302 executes instructions in hazard-recognition program 312 to determine whether a hazard is present based on at least one of the image data 322, the radar detection signal 324, and the lidar detection signal 326. If a hazard exists, hazard detection controller 302 executes instructions in hazard-recognition program 312 to output a hazard-detection signal 236, based on the detected hazard, to system controller 202.

Returning to FIG. 1, if it is determined that a hazard does not exist (No at S106), then the system continues to monitor for hazards (return to S104). However, if it is determined that a hazard exists (Yes at S106), then the hazard is highlighted, and an audio alert is played (S108). This will be described in greater detail with reference to FIG. 2B.

FIG. 2B illustrates the AHDAS 200 at a time ti in accordance with aspects of the present disclosure. As shown in the figure, after receiving hazard-detection signal 236 from hazard-detection system 208 (as discussed above with reference to FIG. 2A), system controller 202 executes instructions in autonomous program 218 to cause system controller 202 to output a HUD-instruction signal 238 to HUD 214 via communication channel 230 and to output a warning-instruction signal 240 to warning system 216 via communication channel 232.

HUD-instruction signal 238 causes HUD 214 to display an image so as to highlight the direction of the hazard. For example, if the hazard is a vehicle in front of the driver's semi-autonomous vehicle and that is rapidly slowing, HUD 214 may display a highlighted box in an area of the windshield that corresponds to a line of sight from the driver to the rapidly slowing vehicle.

Warning-instruction signal 240 causes warning system 216 to play an audio alert to audibly warn the driver of the hazard.

Returning to FIG. 1, after the hazard is highlighted, and an audio alert is played (S108), it is determined whether there is ample time for human response to the hazard (S110). For example, returning to FIG. 2A, system controller 202 may execute instructions in autonomous program 218 to determine whether there is ample time for human response to the hazard.

In some embodiments, memory 204 may include a priori threshold response time data related to a threshold time for which a human can react to a hazard. In general, it may take approximately 200-400 milliseconds for a person's brain to detect a road hazard and another and 400-600 ms for the person's brain to respond to the road hazard. This is dependent on age, training, and an expectation to respond to something coming up. In an example embodiment, 600 milliseconds may be a lower bound for an attentive 25 year old, whereas a full second may be the lower bound for an attentive 65 year old. In some embodiments, this threshold time may be additionally buffered a few hundred milliseconds, wherein approximately 1500 milliseconds may be reasonable minimum threshold to allow a human to detect and respond to a road hazard.

Using hazard-detection signal 236, system controller 202 may execute instructions in autonomous program 218 to determine a time of collision of the semi-autonomous vehicle with the hazard. If the a priori threshold response time data indicates that the threshold time for which a human can react to a hazard is greater than the determined time of collision of the semi-autonomous vehicle with the hazard, then system controller 202 may determine that there is not ample time for human response. Alternatively, if the a priori threshold response time data indicates that the threshold time for which a human can react to a hazard is less than or equal to the determined time of collision of the semi-autonomous vehicle with the hazard, then system controller 202 may determine that there is ample time for human response.

Returning to FIG. 1, if it is determined that there is ample time for human response to the hazard (Yes at S110), then the driver's eyes are tracked, and an EEG analysis is performed (S112).

FIG. 2C illustrates the AHDAS 200 at a time t₂ in accordance with aspects of the present disclosure. Eye-tracking system 210 is configured to output an eye-tracking signal 242 to system controller 202 via communication channel 226, whereas EEG-analyzing system 212 is configured to output a brain-function signal 244 to system controller 202 via communication channel 228.

This will be described in greater detail with reference to FIGS. 4-11. FIG. 4 illustrates a more detailed view of eye-tracking system 210 of AHDAS 200.

As shown in the figure, eye-tracking system 210 includes an eye tracking controller 402, a memory 404, and an internal camera 406. Memory 404 has stored therein data and instructions including an eye-tracking program 408 to be executed by eye tracking controller 402. Eye tracking controller 402 is configured to execute instructions in eye-tracking program 408 of memory 404.

Eye tracking controller 402 may be any device or system that is configured to control general operations of eye-tracking system 210 and includes, but is not limited to, a CPU, a hardware microprocessor, a single core processor, a multi-core processor, an FPGA, a microcontroller, an ASIC, a DSP, or other similar processing device capable of executing any type of instructions, algorithms, or software for controlling the operation and functions of eye-tracking system 210.

Memory 404 may be any device or system capable of storing data and instructions used by eye-tracking system 210 and includes, but is not limited to, RAM, DRAM, a hard drive, a solid-state drive, ROM, EPROM, EEPROM, flash memory, embedded memory blocks in an FPGA, or any other various layers of memory hierarchy.

In this example, eye tracking controller 402, memory 404, and internal camera 406 are illustrated as individual devices of eye-tracking system 210. However, in some embodiments, at least two of eye tracking controller 402, memory 404, and internal camera 406 may be combined as a unitary device. Further, in some embodiments, at least one of eye tracking controller 402, memory 404, and internal camera 406 may be implemented as a computer having non-transitory computer-readable media for carrying or having computer-executable instructions or data structures stored thereon.

Eye-tracking program 408 controls the operations of eye-tracking system 210. Eye-tracking program 408, having a set (at least one) of program modules, may be stored in memory 404 by way of example, and not limitation, as well as an operating system, one or more application programs, other program modules, and program data. Each of the operating system, one or more application programs, other program modules, and program data or some combination thereof, may include an implementation of a networking environment. The program modules generally carry out the functions and/or methodologies of various embodiments of the application as described herein.

As will be described in greater detail below, in some embodiments, eye-tracking program 408 includes instructions, that when executed by eye tracking controller 402, cause eye-tracking system 208 to detect where a driver of the semi-autonomous vehicle is looking and output eye-tracking signal 242 based on where the driver is looking.

Eye tracking controller 402 is configured: to communicate with memory 404 via a communication channel 410; to communicate with internal camera 406 via a communication channel 412; and to communicate with AHDAS controller 202 via communication channel 226.

Internal camera 406 may be any device or system that is configured to image an eye of the driver.

In operation, internal camera 406 transmits eye image data 414 to eye tracking controller 402 via communication channel 412, of an eye, or both eyes, of the driver in real time. The eye image data 414 may take the form of any known image or video standard, non-limiting examples of which include JPEG, PNG, GIF, TIFF, MPR, MOV, MPEG, WMV, AVI, etc.

Eye tracking controller 402 executes instructions in eye-tracking program 408 to determine whether the driver is looking at the hazard. Eye tracking controller 402 further executes instructions in eye-tracking program 409 to cause eye tracking controller to output eye-tracking signal 242 to system controller 202 via communication channel 226.

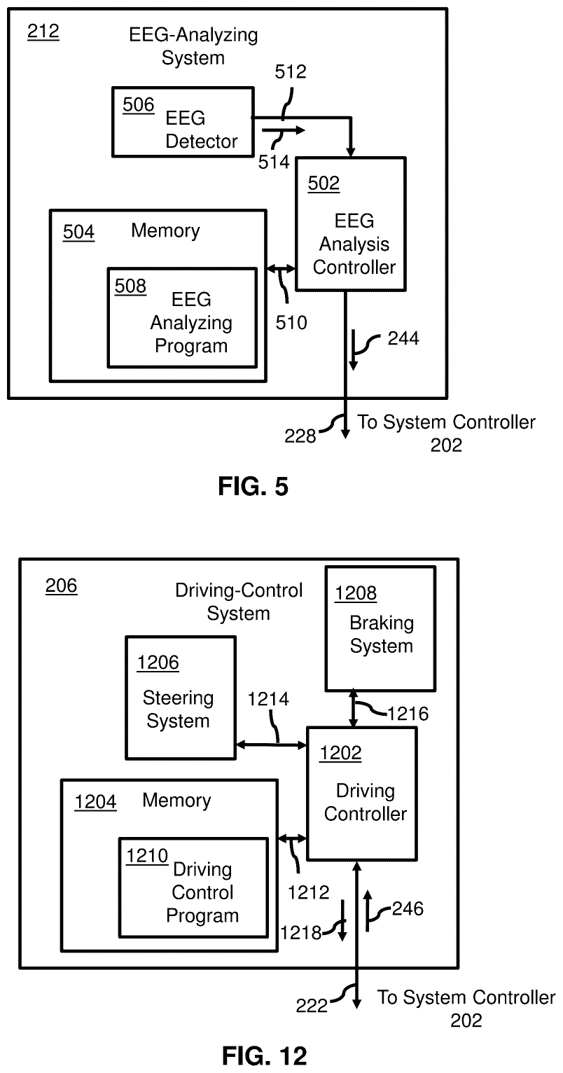

FIG. 5 illustrates a more detailed view of EEG-analyzing system 212 of AHDAS 200.

As shown in the figure, EEG-analyzing system 212 includes an EEG analysis controller 502, a memory 504, and an EEG detector 506. Memory 504 has stored therein data and instructions including an EEG-analyzing program 508 to be executed by EEG analysis controller 502. EEG analysis controller 502 is configured to execute instructions in EEG-analyzing program 508 of memory 504.

EEG analysis controller 502 may be any device or system that is configured to control general operations of EEG-analyzing system 212 and includes, but is not limited to, a CPU, a hardware microprocessor, a single core processor, a multi-core processor, an FPGA, a microcontroller, an ASIC, a DSP, or other similar processing device capable of executing any type of instructions, algorithms, or software for controlling the operation and functions of EEG-analyzing system 212.

Memory 504 may be any device or system capable of storing data and instructions used by EEG-analyzing system 212 and includes, but is not limited to, RAM, DRAM, a hard drive, a solid-state drive, ROM, EPROM, EEPROM, flash memory, embedded memory blocks in an FPGA, or any other various layers of memory hierarchy.

In this example, EEG analysis controller 502, memory 504, and EEG detector 506 are illustrated as individual devices of EEG-analyzing system 212. However, in some embodiments, at least two of EEG analysis controller 502, memory 504, and EEG detector 506 may be combined as a unitary device. Further, in some embodiments, at least one of EEG analysis controller 502, memory 504, and EEG detector 506 may be implemented as a computer having non-transitory computer-readable media for carrying or having computer-executable instructions or data structures stored thereon.

EEG-analyzing program 508 controls the operations of EEG-analyzing system 212. EEG-analyzing program 508, having a set (at least one) of program modules, may be stored in memory 504 by way of example, and not limitation, as well as an operating system, one or more application programs, other program modules, and program data. Each of the operating system, one or more application programs, other program modules, and program data or some combination thereof, may include an implementation of a networking environment. The program modules generally carry out the functions and/or methodologies of various embodiments of the application as described herein.

As will be described in greater detail below, in some embodiments, EEG-analyzing program 508 includes instructions, that when executed by EEG analysis controller 502, cause EEG-analyzing system 212 to detect a parameter of a brain of the driver and output a brain-function signal based on the detected parameter.

As will be described in greater detail below, in some embodiments, EEG-analyzing program 508 includes instructions, that when executed by EEG analysis controller 502, additionally cause EEG-analyzing system 212 to detect a gamma power in a visual cortex part of the brain as the parameter of the brain of the driver.

EEG analysis controller 502 is configured: to communicate with memory 504 via a communication channel 510; to communicate with EEG detector 506 via a communication channel 512; and to communicate with AHDAS controller 202 via communication channel 228.

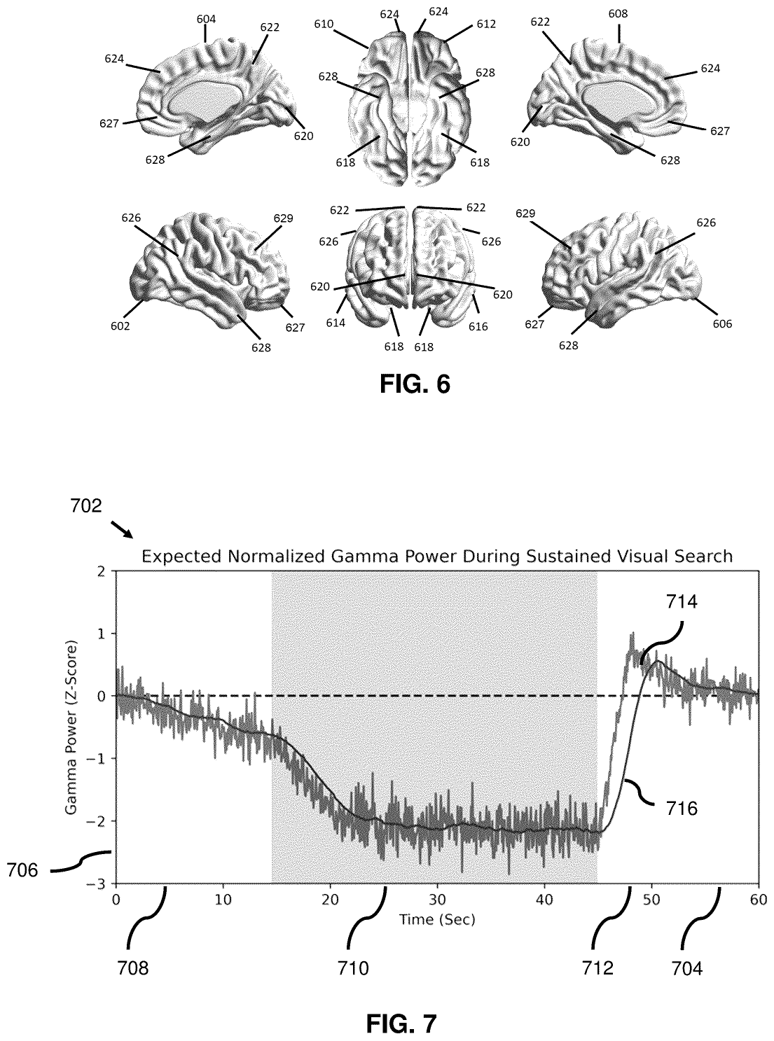

EEG-analyzing system 212 analyzes and interprets electrical activity of the brain of the driver. EEG detector 506 may be any device or system that is configured to detect a parameter of the brain of the driver. In some embodiments, EEG detector 506 detects electrical activity in the brain of the driver. In some embodiments, EEG detector 506 includes electrodes that may be placed on the scalp of the driver, amplifies electrical signals within the brain of the driver and outputs brain function data 514 to EEG analysis controller 502 via communication channel 512. This will be described in greater detail with reference to FIGS. 6-11.

FIG. 6 illustrates multiple views of a human brain. FIG. 6 includes a lateral view 602 of the left hemisphere, a medial view 604 of the left hemisphere, a lateral view 606 of the right hemisphere, a medial view 608 of the right hemisphere, a top view 610 of the left hemisphere, a top view 612 of the right hemisphere, a posterior view 614 of the left hemisphere, and a posterior view 616 of the right hemisphere.

Regions of interest in the brain are indicated as the fusiform gyrus (FG) 618, the primary visual cortex (V1) 620, the precuneus (PC) 622, the dorsomedial prefrontal cortex (DMPFC) 624, the posterior inferior parietal lobule (PIPL) 626, the ventromedial prefrontal cortex (VMPFC) 627, the dorsolateral prefrontal cortex (DLPFC) 629, and the anteromedial temporal cortex (AMT) 628. In example embodiments, VI 620 is a primary area of interest for gamma power detection, as will be discussed in greater detail below. In example embodiments, PC 622, DLPFC 620, and PIPL 626 are areas of interest for a default mode network connectivity strength calculation.

FG 618 is a region of the brain located in the temporal lobe, which is involved in a variety of visual processes, including face recognition, object recognition, and reading. Specifically, FG 618 is known to be particularly important for the perception and recognition of faces. In addition to face recognition, FG 618 is also involved in other types of visual recognition, such as the recognition of words and objects.

In accordance with aspects of the present disclosure, activity in the visual/occipital cortex may be measured. In non-limiting example embodiments, activity in V1 620 is measured. V1 620 is a region of the brain located in the occipital lobe, which is responsible for the initial processing of visual information received from the eyes. Specifically, V1 620 receives visual input from the thalamus, which relays information from the eyes, and processes this information to extract basic features such as color, brightness, and orientation. This initial processing of visual information is known as “early vision.” V1 620 is organized in a highly specialized way, with different regions of the cortex processing information from different parts of the visual field. This organization is known as retinotopy, and it allows the brain to create a detailed map of the visual world. After processing in the primary visual cortex, visual information is sent to other regions of the brain for more complex processing, such as object recognition and spatial navigation.

PC 622 is a region of the brain located in the parietal lobe, which is involved in a variety of cognitive functions, including self-awareness, attention, episodic memory, and visuospatial processing. Research has shown that PC 622 is particularly active during tasks that involve self-reflection and introspection, such as imagining oneself in different situations, thinking about one's personal values and beliefs, and contemplating the future. PC 622 has also been linked to the default mode network (DMN), a network of brain regions that is active when an individual is not engaged in an external task and is instead focused on internal thoughts, including daydreaming and mind-wandering. PC 622 is also involved in spatial awareness and navigation, and it has been shown to be active during tasks that require mental rotation and manipulation of visual images.

DMPFC 624 is a region located in the frontal lobes of the brain, specifically in the dorsomedial (upper and middle) portion. DMPFC 624 is involved in various cognitive, emotional, and social processes. Some key roles that have been associated with DMPFC 624 include self-referential processing, social cognition, social behavior and decision-making, emotional processing, conflict monitoring, and autobiographical memory. With respect to self-referential processing, DMPFC 624 is involved in self-referential processing, including self-referential thinking, self-awareness, and self-relevant decision-making. DMPFC 624 plays a role in constructing and maintaining a coherent sense of self. With respect to social cognition, DMPFC 624 is implicated in social cognition, which involves the processing and interpretation of social information. DMPFC 624 is involved in understanding others' mental states, perspective-taking, empathy, and theory of mind—the ability to attribute mental states to oneself and others. With respect to social behavior and decision-making, DMPFC 624 contributes to social behavior and social decision-making processes. DMPFC 624 is involved in evaluating social rewards and punishments, making judgments about others' trustworthiness, and guiding social interactions. With respect to emotional processing, DMPFC 624 plays a role in emotional processing, including the regulation and integration of emotional experiences. DMPFC 624 is involved in assessing the emotional significance of stimuli and modulating emotional responses. With respect to conflict monitoring, DMPFC 624 is associated with conflict monitoring and resolution. DMPFC 624 helps detect conflicts between competing thoughts, actions, or stimuli and facilitates the resolution of such conflicts. With respect to autobiographical memory, DMPFC 624 is implicated in the retrieval and processing of autobiographical memories—personal memories related to oneself and one's life experiences.

It should be noted that activity in V1 620, PC 622 and DMPFC 624 areas of the brain may be measured to track the dorsal attention network (DAN) activity and the default mode network (DMN) activity in accordance with aspects of the present disclosure, as will be described in greater detail below.

PIPL 626 is a region of the brain located in the parietal lobe, which is involved in a variety of cognitive processes, including attention, perception, and sensorimotor integration. One of the key functions of PIPL 626 is in spatial cognition, which is the ability to perceive, represent, and manipulate spatial information. PIPL 626 is involved in the perception of spatial relationships, including the position of objects in space and the orientation of the body. It is also involved in the control of movements, particularly those that require the integration of sensory and motor information. PIPL 626 is also involved in attentional processes, particularly in tasks that require the allocation of attention to specific stimuli or locations.

VMPFC 627 is a region located in the frontal lobes of the brain, specifically in the ventral and medial aspects. It plays a crucial role in various cognitive and emotional processes, including decision-making, social behavior, emotional regulation, and moral reasoning. Some key functions associated with VMPFC 627 include decision-making, emotional regulation; social behavior; moral reasoning; and self-referential processing.

With respect to decision-making, VMPFC 627 is involved in complex decision-making processes, especially those related to risk and reward. It integrates emotional and cognitive information to assess potential outcomes and guide decision-making. With respect to emotional regulation, VMPFC 627 helps regulate and control emotions by modulating the amygdala, a brain region involved in emotional responses. VMPFC 627 plays a role in evaluating the emotional significance of stimuli and regulating emotional responses accordingly. With respect to social behavior, VMPFC 627 is crucial for social cognition and interpersonal interactions. VMPFC 627 is involved in the processing of social information, such as facial expressions, emotions, and social judgments. Damage to VMPFC 627 can result in changes in social behavior and impaired social decision-making. With respect to moral reasoning, VMPFC 627 contributes to moral decision-making and ethical judgments. VMPFC 627 is involved in evaluating moral dilemmas and integrating emotional and cognitive factors to make moral judgments. With respect to self-referential processing, VMPFC 627 is implicated in self-related processing, including self-referential thinking, self-awareness, and self-evaluation. It plays a role in forming and maintaining a coherent sense of self.

AMT 628 is a region of the brain located in the temporal lobe, which is involved in a variety of cognitive processes, including memory, emotion, and social cognition. One of the key functions of AMT 628 is in the processing of memory, particularly the formation and retrieval of long-term memories. AMT 628 is involved in the consolidation of memories, which is the process by which memories are stabilized and stored in the brain for long-term retention.

DLPFC 629 is a region located in the frontal lobes of the brain, specifically in the dorsolateral (upper and outer) portion. DLPFC 629 plays a critical role in several higher-order cognitive processes and executive functions, including an executive function, working memory, cognitive flexibility, attentional control, inhibitory control, problem-solving and reasoning; and planning and organization. With respect to executive function, DLPFC 629 is involved in executive functions, which are cognitive processes that help plan, organize, initiate, and monitor goal-directed behavior. DLPFC 629 is responsible for working memory, cognitive flexibility, attentional control, and inhibitory control. With respect to working memory, DLPFC 629 is crucial for working memory, which involves the temporary storage and manipulation of information necessary for ongoing cognitive tasks. DLPFC 629 helps maintain relevant information in mind, update it, and use it for decision-making or problem-solving. With respect to cognitive flexibility, DLPFC 629 is involved in cognitive flexibility, which refers to the ability to switch between different mental sets or adapt to changing demands. DLPFC 629 allows individuals to shift attention, change strategies, and adjust behavior based on the situational context. With respect to attentional control, DLPFC 629 plays a role in controlling and maintaining attention. DLPFC 629 helps filter out irrelevant information and focus on relevant stimuli, facilitating sustained attention and selective attention. With respect to inhibitory control, DLPFC 629 is involved in inhibitory control, which refers to the ability to suppress or inhibit inappropriate or impulsive responses. DLPFC 629 helps regulate behavior, inhibit automatic responses, and make decisions based on long-term goals rather than immediate impulses. With respect to problem-solving and reasoning, DLPFC 629 contributes to problem-solving, logical reasoning, and abstract thinking. DLPFC 629 helps analyze complex information, generate hypotheses, and plan and execute strategies to solve problems. With respect to planning and organization, DLPFC 629 is involved in planning and organizing actions towards a specific goal. DLPFC 629 helps break down tasks into subgoals, sequence actions, and allocate cognitive resources efficiently.

By analyzing electrical activity through one or more of the above discussed regions of the brain of the driver, AHDAS 200 may determine whether the driver comprehends the hazard. In particular, sustained visual attention leads to decreased gamma power in VI 620. This will be described in greater detail with reference to FIG. 7.

FIG. 7 illustrates sustained gamma power graphs of a human brain. Gamma power and gamma z-score are measures used to analyze brain function, particularly in the context of EEG.

Gamma power refers to the strength or amplitude of gamma frequency (30-200 Hz) oscillations in the brain. Gamma oscillations are believed to be involved in a variety of cognitive processes, including attention, memory, and perception, and their strength or power has been linked to cognitive performance.

Gamma z-score, on the other hand, is a signal normalization technique applied to signals detected within an individual. Over long time periods, the mean of the z-scored signal is zero and the standard deviation of the signal fluctuations is 1. The z-score normalizes the gamma signals for consistent processing.

FIG. 7 includes: a gamma power graph 702 for a V1 620 of a user having sustained visual attention over a period of 64 seconds. Gamma power graph 702 includes an x-axis 704 in time from 0-60 seconds and a y-axis 706 in z-score from −3 to 3. Further, gamma power graph 702 includes a resting portion 708 from 0-15 seconds wherein a user is resting, a task portion 710 from 15-45 seconds wherein a user must focus on performing a task, and a resting portion 712 from 45-60 seconds wherein the user is again resting. Gamma power graph 702 includes a gamma power function 714 and an average (smoothed) gamma power function 716.

It should be noted that there is a drop in gamma power function 714 during task portion 710. The sustained decrease in gamma power in is an indicator of attention to the external environment.

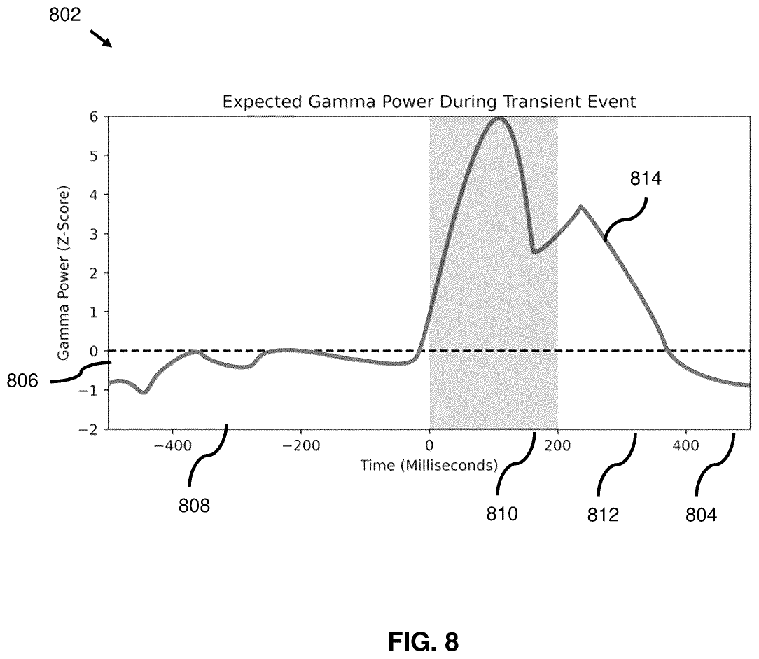

However, individual stimuli related to a visual attention task induce transient spikes in gamma power when presented. Similar signals can be evaluated in a driving scenario to determine recognition of a road hazard highlighted on a HUD. This will be described in greater detail with reference to FIG. 8.

FIG. 8 illustrates a convolved gamma power graph of a human brain. Convolved gamma power is a measure used to analyze brain function. It is a method that combines two signals—the EEG signal and a stimulus signal—to investigate the neural activity that is specifically related to the processing of the stimulus. The convolved gamma power measure involves convolving the EEG signal with a waveform that represents the timing of the stimulus. This process effectively aligns the EEG signal with the onset of the stimulus and allows for the extraction of neural activity that is specifically related to the processing of the stimulus. After convolution, the signal is analyzed for gamma power, which is the strength or amplitude of gamma frequency (30-200 Hz) oscillations in the brain. Convolved gamma power can be used to investigate the neural activity that is specifically related to the processing of a stimulus.

FIG. 8 includes: a convolved gamma power graph 802 for a V1 620 of a user having a visual stimulus for a period of 200 microseconds.

Convoluted gamma power graph 802 includes an x-axis 804 in time from −500 to 500 microseconds and a y-axis 806 in z-score from −2 to 6. Further, convoluted gamma power graph 802 includes: a non-stimulating portion 808 from −500-0 microseconds, wherein a user is not experiencing any visual stimulation; a stimulation portion 810 from 0-200 microseconds, wherein a user experiences a visual stimulus; and a non-stimulating portion 812 from 200-500 microseconds, wherein the user is again not experiencing any visual stimulation. Convoluted gamma power graph 802 additionally includes a convoluted gamma power function 814.

It should be noted, as evidenced by convoluted gamma power graph 802, there is a dramatic transient spike in the convoluted gamma power function during the stimulation portion for V1 620. The transient spikes in convoluted gamma power graph 802 is an indicator of a detection of something novel that grabs the attention of the driver.

Furthermore, the dorsal attention network (DAN) is a collection of brain regions centered on the intraparietal sulcus and the frontal eye fields.

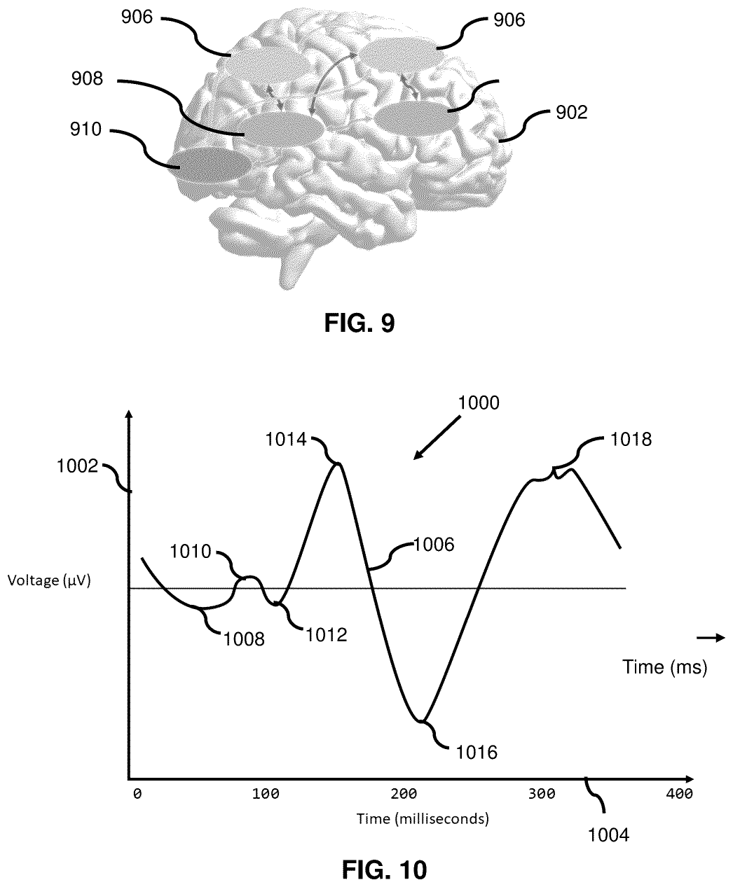

FIG. 9 illustrates a dorsal attention network, a ventral attention network, and a visual cortex of a human brain. The figure includes a lateral view of a right hemisphere 902 of a brain. The brain includes a dorsal attention network (DAN) 906, a ventral attention network (VAN) 908, and a visual cortex 910.

DAN 906 is a brain network that is involved in spatial attention and the allocation of attentional resources. It includes regions such as the intraparietal sulcus and the superior parietal lobule, which are located in the posterior part of the brain. DAN 906 is activated when a person needs to focus attention on a particular location in space, such as when searching for a specific object in a cluttered environment. It is also involved in the coordination of eye and hand movements, which is important for tasks such as reaching and grasping. The network receives input from sensory regions of the brain, such as visual cortex 910.

VAN 908 is a brain network that is involved in detecting and responding to salient stimuli in the environment. It includes regions such as the anterior insula and FG 618, which are located in the frontal and temporal lobes of the brain. VAN 908 is activated when a person encounters unexpected or behaviorally relevant stimuli, such as a sudden loud noise or the appearance of a new object in our environment.

Visual cortex 910 is a part of the brain that is responsible for processing visual information from the eyes. It is located in the occipital lobe at the back of the brain. When light enters the eye, it is detected by specialized cells called photoreceptors in the retina. The photoreceptors then send signals through the optic nerve to the visual cortex, where the information is processed to create our perception of the visual world. The visual cortex is organized into a series of distinct regions that are specialized for different aspects of visual processing. For example, V1 620 is responsible for basic visual processing such as edge detection and orientation tuning. Other regions, such as the ventral stream and the dorsal stream, are responsible for more complex visual processing, such as object recognition, spatial awareness, and motion perception.

Activity of any of DAN 906, VAN 908 or visual cortex 910 relate to an individual orienting their focus to a task. DAN 906 is also active when focus is directed towards an object. When an individual directs their focus externally on an object, brain signals collected from DAN 906 will be highly coherent, or statistically correlated. Furthermore, the signals from DAN 906 are anti-correlated with regions belonging to the default mode network (DMN), which includes brain regions important for episodic memory that are highly active and correlated when an individual is daydreaming or not focusing intently on the external world.

Therefore, when a semi-autonomous vehicle detects a hazard, this system also correlates EEG signals between DAN regions to form a network connectivity strength metric, the same calculation is done between DMN regions. If a driver has directed their gaze towards the hazard and DAN connectivity strength is high while DMN connectivity strength is low, the semi-autonomous vehicle will consider that the driver is alert to the external world and sufficiently processing the object of their visual focus. If DAN connectivity strength is low and DMN strength is high, then the semi-autonomous vehicle will consider that the driver is attending to their internal thoughts more than the external world. The semi-autonomous vehicle will continue to monitor for DAN connectivity strength increases as long as there is ample time for the driver to respond to the hazard.

In accordance with aspects of the present disclosure, part of the evaluation of driver attention includes Event-Related Potential (ERP) analysis on brain wave patterns that arise due to stimulus events. ERPs are positive or negative voltage displacements in the EEG signal that occur in response to sensory processing of novel stimuli by the brain. ERPs have a temporal resolution that allows for measurement of brain activity from one millisecond to the next and are well suited to evaluate attention as perceptual and attentional processes may evolve over the course of tens of milliseconds. This will be described in greater detail with reference to FIG. 10.

FIG. 10 illustrates a graph 1000 of voltage displacements of an EEG signal related to novel stimulus processing. As shown in the figure, graph 1000 includes a y-axis 1002 of voltage in microvolts, an x-axis 1004 of time in milliseconds, and a function 1006. Function 1006 includes a local minimum 1008, a local peak 1010, a local minimum 1012, a local peak 1014, a local minimum 1016, and a local peak 1018.

Function 1006 represents a brainwave associated with a reaction to a novel stimulus, as detected by EEG detector 506. The series of voltage fluctuations of local minimum 1008, local peak 1010, local minimum 1012, local peak 1014, local minimum 1016, and local peak 1018 correspond to the brain transforming raw sensory input into a cognitive percept that may be followed by a behavioral response. Specifically, local minimum 1008, local peak 1010 and local minimum 1012 occur as information propagates through the visual system and a perception is formed. Local peak 1014 and local minimum 1016 correspond to categorization of the stimulus. Finally, the local peak 1018 following EEG signal relates to working memory encoding and maintenance of the perception. These voltage differences may be followed by signal components related to the preparation of a motor response to the stimulus (lateralized-readiness potential, or LRP, not shown), and then signal components that occur during the behavioral response. Features of the stimulus (e.g., color, shape, size) can modulate ERP component amplitudes.

Signal polarity also relates to the location where a stimulus first enters the visual field. Accordingly, ERP analysis may be used in conjunction with information relating to the visual characteristics of a hazard to evaluate if the transient brain signal is associated with the hazard. For example, if features of the stimulus do not appear to have modulated expected ERP components, AHDAS 200 may determine that the driver has not sufficiently detected the hazard.

Returning to FIG. 2C, system controller 202 may execute instructions in autonomous program 218 to cause system controller 202 to consider multiple timepoints as the stimulus onset and evaluate the timing of ERP components for all candidate possibilities. In some embodiments, system controller 202 may execute instructions in autonomous program 218 to cause system controller 202 to consider potential stimulus onset times as: 1) the moment when the hazard is first within the fovea of the driver, which may be based on eye-tracking signal 242 from eye-tracking system 210; 2) the moment when AHDAS 200 first highlights the hazard on HUD 214 and concurrently plays an audio alert on warning system 216 (see S108 above); and 3) the first moment that the hazard is anywhere in the driver's visual field, which may be based on eye-tracking signal 242. It should be noted that the fovea is a small central area of the retina in the eye that is responsible for sharp and detailed vision. When an object is within a person's fovea, it means that the object is located in the very center of their visual field and they are able to see it with high visual acuity and detail. On the other hand, the visual field refers to the entire area that a person can see while their eyes are fixed in a certain position, including both their central and peripheral vision. This means that an object can be within a person's visual field without necessarily being in their fovea, and therefore may not be seen with the same level of detail and clarity.

Returning to FIG. 5, memory 504 may include a library of a priori ERPs corresponding to recognition by people of hazards. Further, as will be described in greater detail below, this library of a priori ERPs may be updated and used to train EEG-analyzing program 508 such that EEG analysis controller 502 may execute EEG-analyzing program 508 to determine whether a newly detected ERP corresponds to a driver recognizing a hazard. The data of the components of the ERP may then be output to system controller 202 by way of brain-function signal 244.

Returning to FIG. 2C, in some example embodiments, system controller 202 may execute instructions in autonomous program 218 to cause system controller 202 to evaluate the 600 milliseconds of EEG data that follow the onset times to detect components of the ERP.

If system controller 202 detects an ERP that corresponds to one of the candidate stimulus onset times, then system controller 202 may execute instructions in autonomous program 218 to cause system controller 202 to determine that the driver has sufficiently processed the hazard. In some embodiments, in addition to evaluating ERPs, part of the EEG attention evaluation can include analysis of pre-stimulus measures. Brain activity one-to-three seconds before a specific visual stimulus can inform how well the driver will process the stimulus. As previously mentioned, eye-tracking gaze measures can determine the timing of pre-stimulus signals vs post-stimulus signals. For example, the time separating pre-stimulus and post-stimulus signals may be based on when a hazard is determined to be within the fovea of the eye. Based on the pre-stimulus signal, predictions may be made as to comprehension and reaction time with respect to the stimulus. Such pre-stimulus analysis may be based on spectral measures (specific frequency bands), functional connectivity (signal coherence between brain regions), or both. With respect to spectral measures, for instance, the visual cortex EEG amplitude in response to a presented stimulus is related to pre-stimulus alpha-power. When pre-stimulus alpha band power is high, greater amplitudes between 1008 and 1012 will be present in the post-stimulus signal.

ERP amplitudes between 1008 and 1012 after novel stimulus onset are related to alpha-band (8-12 Hz) power prior to the stimulus. Low-alpha power prior to the semi-autonomous vehicle highlighting a road hazard on the HUD may indicate that a driver will comprehend the road hazard too slowly to react to it.

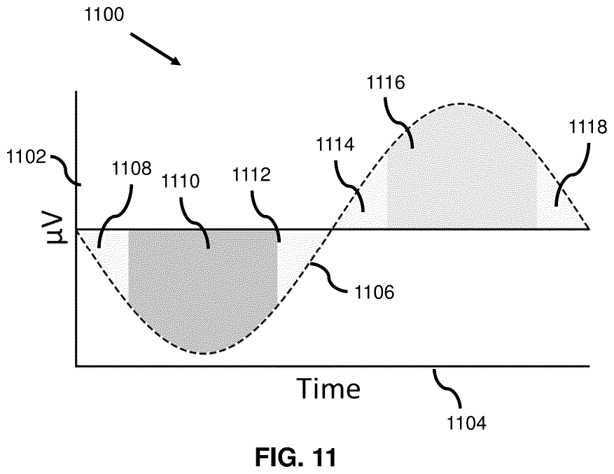

Further, the phase of an EEG signal at alpha and theta frequencies in the frontoparietal network (also known as the executive control network or central executive network) at stimulus onset are indicative of how quickly an individual may respond to the stimulus. This will be described in greater detail with reference to FIG. 11.

FIG. 11 illustrates a signal phase of a brain network. FIG. 11 includes a y-axis 1102 in microvolts, an x-axis 1104 in milliseconds, and a signal 1106. Signal 1106 may be broken down into sections 1108, 1110, 1112, 1114, 1116, and 1118. In sections 1108, 1112, 1114, and 1118, the detected microvolts indicate an average reaction time if a stimulus occurs during any of these periods. Section 1110 indicates a slow reaction time if a stimulus occurs during this period. Section 1116 indicates a quick reaction time if a stimulus occurs during this period. Brain networks related to internal focus (e.g., memories) and external focus (e.g., visual attention) may be monitored. Returning to FIG. 2, system controller 202 may execute instructions in autonomous program 218 to cause system controller 202 to analyze the signal at the moment a hazard is highlighted on the HUD to estimate how much time it will take the human driver to process the road hazard. In particular, if the moment a hazard is highlighted on the HUD corresponds to any of sections 1108, 1112, 1114, and 1118, then system controller 202 may determine that the user may have an average reaction time. For example, depending on age, the average time to detect and respond to a hazard could range from 600 ms to 1500 ms, wherein 600 ms may be the average time for younger drivers and 1500 ms may be the average for older drivers. If we want we can just go with the larger number and say 1500 ms.

Further, if the moment a hazard is highlighted on the HUD corresponds to section 1110, then system controller 202 may determine that the user may have a slow reaction time, i.e., greater than average. Still further, if the moment a hazard is highlighted on the HUD corresponds to section 1116, then system controller 202 may determine that the user may have a fast reaction time, i.e., less than average.

Memory 204 may have an a priori driver hazard processing threshold stored therein. System controller 202 may execute instructions in autonomous program 218 to cause system controller 202 to compare the estimated time it will take the driver to process the road hazard with the priori driver hazard processing threshold. If the estimated time it will take the driver to process the road hazard is less than the priori driver hazard processing threshold, then system controller 202 may determine that the human reaction time will be sufficient to react to the hazard. Alternatively, if the estimated time it will take the driver to process the road hazard is equal to or greater than the priori driver hazard processing threshold, then system controller 202 may determine that the human reaction time will be too slow based on signal phase analysis. As such, as will be discussed in greater detail below, AHDAS 200 will enter an autonomous mode to deal with the impending road hazard.

Finally, part of the attention evaluation can include EEG-based classification, where deep learning models use EEG signals to predict what an individual saw. Typically, EEG-based inputs to the deep learning model will be composed of whole-brain activity maps, whole-brain connectivity maps (seed-based or latent-component based), or wavelet transforms of the EEG signals.

During algorithm training, EEG data may be collected from a large group of individuals looking at various objects or scenes (in practice the training images could include scenes of accidents on the side of the road or video of a rapidly decelerating vehicle directly in front of the driver). Raw signals are converted to various brain activity and functional connectivity maps. The algorithm may then be trained to predict what object or scenario an individual was looking at based on their brain activity and connectivity maps when the object or scene was in their visual field.

For 2D image data, the algorithm could be a CNN; but the algorithm could be a hybrid CNN-LSTM for video data. Vehicles in production would have a trained version of this algorithm on-board. The semi-autonomous vehicle could then continuously monitor EEG data and use this algorithm to determine what objects, scenes, or hazards, the driver's brain is processing and comprehending in real-time.

Accordingly, the semi-autonomous vehicle may determine based on EEG-based classification that a driver has or has not detected a hazard based on the prediction of the deep-learning model. Furthermore, the semi-autonomous vehicle may determine that a driver misperceived a hazard. For example, the EEG-based classification may predict that the driver operating the semi-autonomous vehicle at night perceived the taillights of a motorcycle ahead as those of a more distant truck or car. In such a situation, the semi-autonomous vehicle may determine that the driver has incorrectly identified the hazard.

Returning to FIG. 1, after the driver's eyes are tracked and an EEG analysis is performed (S112), it is determined whether the driver comprehended the hazard (S114). For example, returning to FIG. 2C, system controller 202 may execute instructions in autonomous program 218 to cause system controller 202 to: determine whether there is a hazard, and if so, the location of the hazard based on data within hazard-detection signal 236 from hazard-detection system 208 (see FIG. 2A); determine whether the driver is viewing the hazard based on data within the eye-tracking signal 242 from eye-tracking system; and determine whether the driver comprehended the hazard based on the brain-function signal 244 from EEG-analyzing system 212.

Returning to FIG. 1, if it is determined that the driver did not comprehend the hazard (No at S114), then it is again determined whether there is ample time for human response (return to S110). For example, as shown in FIG. 2C, if system controller 202 determines at least one of: there is no hazard based on data within hazard-detection signal 236 from hazard-detection system 208 (see FIG. 2A); the driver is not viewing the hazard based on data within the eye-tracking signal 242 from eye-tracking system; and the driver does not comprehend the hazard based on the brain-function signal 244 from EEG-analyzing system 212, then system controller 200 determines that the driver did not comprehend the hazard.

However, if it is determined that the driver did comprehend the hazard (Yes at S114), then the steering and braking behavior are monitored (S116). For example, as shown in FIG. 2C, if system controller 202 determines: the location of the hazard based on data within hazard-detection signal 236 from hazard-detection system 208 (see FIG. 2A); the driver is viewing the hazard based on data within the eye-tracking signal 242 from eye-tracking system; and the driver does comprehend the hazard based on the brain-function signal 244 from EEG-analyzing system 212, then system controller 200 monitors steering and braking behavior via driving-control system 206.

In an example embodiment, system controller 200 executes instructions in autonomous program 218 to cause system controller 202 to output a driving monitoring signal 246 to driving-control system 206. Driving monitoring signal 246 instructs driving-control system 206 to monitor the steering and braking of the semi-autonomous vehicle. This will be described in greater detail with reference to FIG. 12.

FIG. 12 illustrates a more detailed view of driving-control system 206 of AHDAS 200.

As shown in the figure, driving-control system 206 includes a driving controller 1202, a memory 1204 a steering system 1206, and a braking system 1208. Memory 1204 has stored therein data and instructions including a driving-control program 1210 to be executed by driving controller 1202. Driving controller 1202 is configured to execute instructions in driving-control program 1210 of memory 1204.

Driving controller 1202 may be any device or system that is configured to control general operations of driving-control system 206 and includes, but is not limited to, a CPU, a hardware microprocessor, a single core processor, a multi-core processor, an FPGA, a microcontroller, an ASIC, a DSP, or other similar processing device capable of executing any type of instructions, algorithms, or software for controlling the operation and functions of driving-control system 206.

Memory 1204 may be any device or system capable of storing data and instructions used by driving-control system 206 and includes, but is not limited to, RAM, DRAM, a hard drive, a solid-state drive, ROM, EPROM, EEPROM, flash memory, embedded memory blocks in an FPGA, or any other various layers of memory hierarchy.

Steering system 1206 may be any device or system that is configured to: monitor the steering of the semi-autonomous vehicle; and operate in: a driver-controlled state so as to enable the driver to control steering of the semi-autonomous vehicle; and a driving-control-system-controlled state so as to enable driving-control system 206 to control the steering of the semi-autonomous vehicle. In some embodiments, by default, steering system 1206 is configured to operate in the driver-controlled state.

Braking system 1208 may be any device or system that is configured to: monitor the braking of the semi-autonomous vehicle; and operate in: a driver-controlled state so as to enable the driver to control braking of the semi-autonomous vehicle; and a driving-control-system-controlled state so as to enable driving-control system 206 to control the breaking of the semi-autonomous vehicle. In some embodiments, by default, braking system 1208 is configured to operate in the driver-controlled state.

In this example, driving controller 1202, memory 1204, steering system 1206, and braking system 1208 are illustrated as individual devices of driving-control system 206. However, in some embodiments, at least two of controller 1202, memory 1204, steering system 1206, and braking system 1208 may be combined as a unitary device. Further, in some embodiments, at least one of controller 1202, memory 1204, steering system 1203, and braking system 1208 may be implemented as a computer having non-transitory computer-readable media for carrying or having computer-executable instructions or data structures stored thereon.

Driving-control program 1210 controls the operations of driving-control system 206. Driving-control program 1210, having a set (at least one) of program modules, may be stored in memory 1204 by way of example, and not limitation, as well as an operating system, one or more application programs, other program modules, and program data. Each of the operating system, one or more application programs, other program modules, and program data or some combination thereof, may include an implementation of a networking environment. The program modules generally carry out the functions and/or methodologies of various embodiments of the application as described herein.

As will be described in greater detail below, in some embodiments, driving-control program 1210 includes instructions, that when executed by driving controller 1202, cause driving-control system 206 to determine whether the driver comprehends the hazard based on the hazard-detection signal, the eye-tracking signal and the brain-function signal; and operate in: a driver-controlled state so as to enable the driver to control at least one of steering of the semi-autonomous vehicle, breaking of the semi-autonomous vehicle, and a combination thereof; and a driving-control-system-controlled state so as to enable the driving-control system to control the at least one of steering of the semi-autonomous vehicle, breaking of the semi-autonomous vehicle, and a combination thereof.

As will be described in greater detail below, in some embodiments, driving-control program 1210 includes instructions, that when executed by driving controller 1202, cause driving-control system 206 additionally to determine whether the semi-autonomous vehicle has cleared the hazard; and operate in the driver-controlled state if the semi-autonomous vehicle has cleared the hazard and when previously operating in the driving-control-system-controlled state.

Driving controller 1202 is configured: to communicate with memory 1204 via a communication channel 1212; to communicate with steering system 1206 via a communication channel 1214; to communicate with braking system 1208 via a communication channel 1216; and to communicate with AHDAS controller 202 via communication channel 222.

In operation, upon receiving driving monitoring signal 246, driving controller 1202 executes instructions in driving-control program 1210 to cause driving controller 1202 to monitor the steering of the semi-autonomous vehicle via steering system 1206 and to monitor the braking of the semi-autonomous vehicle via braking system 1208. Driving controller 1202 outputs a steering/braking signal 1218 to system controller 202 via communication channel 222. Steering/braking signal 1218 includes information describing the amount of braking and the direction of steering of the semi-autonomous vehicle by the driver.

Returning to FIG. 1, after the steering and braking behavior are monitored (S116), it is determined whether appropriate decisions are made (S118). This will be described in greater detail with reference to FIG. 2E.

FIG. 2E illustrates the AHDAS 200 at a time t₄ in accordance with aspects of the present disclosure. As shown in the figure, hazard-detection system 208 transmits an updated hazard-detection signal 248, which updates the location and speed of the hazard. In should be noted that in some embodiments, hazard-detection system 208 may output a hazard-detection signal periodically, for example every 10 milliseconds.

Upon receiving steering/braking signal 1218 from driving control system 206 and updated hazard-detection signal 248 from hazard-detection system 208, system controller 202 executes instructions in autonomous program 218 to determine whether the drivers braking and/or steering are appropriate decisions in that the hazard will be avoided.

Returning to FIG. 1, if it is determined that appropriate decisions are made (Yes at S118), then it is determined whether the hazard is cleared (S120). This will be described in greater detail with reference to FIG. 2F.

FIG. 2F illustrates the AHDAS 200 at a time t₅ in accordance with aspects of the present disclosure. As shown in the figure, hazard-detection system 208 transmits an updated hazard-detection signal 250, which updates the location and speed of the hazard, to system controller 202 via communication channel 224. Further driving control system 206 transmits an updated steering/braking signal 252, detailing the steering and braking of the driver, to system controller 202 via communication channel 222.

Upon receiving updated steering/braking signal 252 from driving control system 206 and updated hazard-detection signal 250 from hazard-detection system 208, system controller 202 executes instructions in autonomous program 218 to determine whether the hazard has been cleared.

Returning to FIG. 1, if it is determined that the hazard has not been cleared (No at S120), then the steering and braking behaviors are again monitored (return to S116). However, if it is determined that the hazard has been cleared (Yes at S120), then the driver continues normal vehicle operation (S122). For example, returning to FIG. 6, steering system 1206 and braking system 1208 each continue to operate in a driver-controlled state, wherein the driver controls the steering and braking of the semi-autonomous vehicle.

Returning to FIG. 1, after the driver continues normal vehicle operation (S122), process 100 stops (S124). In other words, during this portion of process 100, in this example: AHDAS 200 determined that there was a hazard (Yes at S106); AHDAS 200 provided a warning and HUD alert to the driver (S108); AHDAS 200 determined that there was time for the driver to respond to the hazard (Yes at S110); AHDAS 200 tracked the driver's eyes and performed an EEG analysis on the driver's brain (S112); AHDAS 200 determined that the driver comprehended the hazard (Y at S114); AHDAS 200 monitored the steering and braking of the semi-autonomous vehicle (S116); AHDAS 200 determined that the driver had taken appropriate actions to avoid the hazard (Yes at S118); AHDAS 200 determined that the hazard was cleared (Yes at S 120); and finally, AHDAS 200 enabled the driver to continue normal vehicle operation (S122).

If it is determined that appropriate decisions are not made (No at S118), then a message is displayed and drive functions are autonomously controlled (S126). For example, if the driver does not break enough or does not steer the semi-autonomous vehicle so as to avoid the hazard, then AHDAS 200 will take further action. This will be described in greater detail with reference to FIG. 2G.

FIG. 2G illustrates the AHDAS 200 at a time t₆ in accordance with aspects of the present disclosure. As shown in the figure, system controller system 202 executes instructions in autonomous program 218 to cause system controller 202 to output a HUD-instruction signal 254 to HUD 214 via communication channel 230. Further, system controller 202 executes instructions in autonomous program 218 to cause system controller 202 to output a warning-instruction signal 256 to warning system 216 via communication channel 232. Still further, system controller 202 executes instructions in autonomous program 218 to cause system controller 202 to output a driving-control signal 258 to driving control system 206 via communication channel 222.

HUD-instruction signal 254 causes HUD 214 to display an image indicating an autonomous control of steering/braking, which informs the driver that AHDAS 200 is temporarily taking control of steering/breaking of the semi-autonomous vehicle. Warning-instruction signal 256 causes warning system 216 to play an audio alert to audibly warn the driver that AHDAS 200 is temporarily taking control of steering/breaking of the semi-autonomous vehicle. Driving-control signal 258 causes steering system 1206 (as shown in FIG. 6) to operate in the driving-control-system-controlled state so as to enable driving-control system 206 to control the steering of the semi-autonomous vehicle and causes braking system 1208 to operate in the driving-control-system-controlled state so as to enable driving-control system 206 to control the braking of the semi-autonomous vehicle. In the driving-control-system-controlled state, driving controller 1202 executes instructions in driving control program 1210 to cause steering system 1206 to steer the semi-autonomous vehicle and to cause braking system 1208 to brake the semi-autonomous vehicle so as to avoid the hazard.

Returning to FIG. 1, after a message is displayed and drive functions are autonomously controlled (S126), it is determined whether the hazard is cleared (S128). This may be performed in a manner similar to that as discussed above (see S120). If it is determined that the hazard has not been cleared (No at S128), then the message is continued to be displayed and drive functions continue to be autonomously controlled (return to S126). However, if it is determined that the hazard has been cleared (Yes at S128), then control is returned to the driver (S130). This will be described in greater detail with reference to FIG. 2H.

FIG. 2H illustrates the AHDAS 200 at a time t₇ in accordance with aspects of the present disclosure. As shown in the figure, system controller system 202 executes instructions in autonomous program 218 to cause system controller 202 to a driving-control signal 260 to driving control system 206 via communication channel 222.

Driving-control signal 260 causes steering system 1206 (as shown in FIG. 6) to operate in the driver-controlled state so as to enable the driver to control steering of the semi-autonomous vehicle and causes braking system 1208 to operate in the driver-controlled state so as to enable the driver to control braking of the semi-autonomous vehicle.

Returning to FIG. 1, after control is returned to the driver (S130), then the driver continues normal vehicle operation (S122) and process 100 stops (S124).

In summary, if it AHDAS 200 determines that appropriate decisions are not made to avoid the hazard (No at S118), then AHDAS 200 warns the driver that AHDAS 200 will be temporarily taking control of the semi-autonomous vehicle and takes control of steering and braking of the semi-autonomous vehicle (S126) until the hazard is cleared (S128). At that point, AHDAS 200 returns control the driver (S130).

The foregoing description of various preferred embodiments have been presented for purposes of illustration and description. It is not intended to be exhaustive or to limit the invention to the precise forms disclosed, and obviously many modifications and variations are possible in light of the above teaching. The example embodiments, as described above, were chosen and described in order to enable others skilled in the art to best utilize the invention in various embodiments and with various modifications as are suited to the particular use contemplated. It is intended that the scope of the invention be defined by the claims appended hereto.

What is claimed is:

1. A vehicle control system for use with a semi-autonomous vehicle, said vehicle control system comprising:
a hazard-detection system comprising:
a hazard-detection memory having hazard-detection instructions stored therein; and
a hazard-detection processor configured to execute the hazard-detection instructions to cause said hazard-detection system to:
detect a hazard; and
output a hazard-detection signal based on the detected hazard;
an eye-tracking system comprising:
an eye-tracking memory having eye-tracking instructions stored therein; and
an
eye-tracking processor configured to execute the eye-tracking instructions to cause the eye-tracking system to:
detect where a driver of the semi-autonomous vehicle is looking; and
output an eye-tracking signal based on where the driver is looking;
a brain-function system comprising:
a brain-function memory having brain-function instructions stored therein; and
a brain-function processor configured to execute the brain-function instructions to cause the brain-function system to:
detect a parameter of a brain of the driver; and
output a brain-function signal based on the detected parameter; and
a driving-control system comprising:
a driving-control memory having driving-control instructions stored therein; and
a driving-control processor configured to execute the driving-control instructions to cause the driving-control system to:
determine whether the driver comprehends the hazard based on the hazard-detection signal, the eye-tracking signal and the brain-function signal; and
operate in: a driver-controlled state so as to enable the driver to control at least one of steering of the semi-autonomous vehicle, breaking of the semi-autonomous vehicle, and a combination thereof; and a driving-control-system-controlled state so as to enable the driving-control system to control the at least one of steering of the semi-autonomous vehicle, breaking of the semi-autonomous vehicle, and a combination thereof.

2. The vehicle control system of claim 1, further comprising a warning system configured to provide a warning related to said driving-control system controlling the at least one of steering of the semi-autonomous vehicle, breaking of the semi-autonomous vehicle, and a combination thereof when said driving-control system is operating in the driving-control system state.

3. The vehicle control system of claim 2, wherein the warning system comprises a display configured to display an image related to said driving-control system controlling the at least one of steering of the semi-autonomous vehicle, breaking of the semi-autonomous vehicle, and a combination thereof when said driving-control system is operating in the driving-control system state.

4. The vehicle control system of claim 3, wherein said display comprises a head up display.

5. The vehicle control system of claim 2, wherein said warning system comprises a speaker configured to output an audible warning related to said driving-control system controlling the at least one of steering of the semi-autonomous vehicle, breaking of the semi-autonomous vehicle, and a combination thereof when said driving-control system is operating in the driving-control system state.

6. The vehicle control system of claim 1, wherein said driving-control processor is further configured to execute the driving-control instructions to additionally cause said driving-control system to:
determine whether the semi-autonomous vehicle has cleared the hazard; and
operate in the driver-controlled state if the semi-autonomous vehicle has cleared the hazard and when previously operating in the driving-control-system-controlled state.

7. The vehicle control system of claim 1, wherein said brain-function processor is further configured to execute the brain-function instructions to cause said brain-function system to detect a gamma power in a visual cortex part of the brain as the parameter of the brain of the driver.

8. A method of operating a semi-autonomous vehicle, said method comprising:
detecting, via hazard-detection processor configured to execute hazard-detection instructions stored in a hazard-detection memory, a hazard;
outputting, via the hazard-detection processor, a hazard-detection signal based on the detected hazard;
detecting, via an eye-tracking processor configured to execute eye-tracking instructions stored in an eye-tracking memory, where a driver of the semi-autonomous vehicle is looking;
outputting, via the eye-tracking processor, an eye-tracking signal based on where the driver is looking;
detecting, via a brain-function processor configured to execute brain-function instructions stored in a brain-function memory, a parameter of a brain of the driver;
outputting, via the brain-function processor, a brain-function signal based on the detected parameter;
determining, via a driving-control processor configured to execute driving-control instructions stored in a driving-control memory, whether the driver comprehends the hazard based on the hazard-detection signal, the eye-tracking signal and the brain-function signal; and
operating the driving-control processor in: a driver-controlled state so as to enable the driver to control at least one of steering of the semi-autonomous vehicle, breaking of the semi-autonomous vehicle, and a combination thereof; and a driving-control-system-controlled state so as to enable the driving-control system to control the at least one of steering of the semi-autonomous vehicle, breaking of the semi-autonomous vehicle, and a combination thereof.

9. The method of claim 8, further comprising providing, via a warning system, a warning related to the driving-control system controlling the at least one of steering of the semi-autonomous vehicle, breaking of the semi-autonomous vehicle, and a combination thereof when the driving-control system is operating in the driving-control system state.

10. The method of claim 9, wherein said comprising a warning comprises displaying, via a display, an image related to the driving-control system controlling the at least one of steering of the semi-autonomous vehicle, breaking of the semi-autonomous vehicle, and a combination thereof when the driving-control system is operating in the driving-control system state.

11. The method of claim 10, wherein said displaying the image comprises displaying the image via a head up display.

12. The method of claim 9, wherein said comprising a warning comprises outputting, via a speaker, an audible warning related to the driving-control system controlling the at least one of steering of the semi-autonomous vehicle, breaking of the semi-autonomous vehicle, and a combination thereof when the driving-control system is operating in the driving-control system state.

13. The method of claim 8, further comprising:
determining, via the driving-control processor, whether the semi-autonomous vehicle has cleared the hazard; and
operating, via the driving-control processor, in the driver controlled state if the semi-autonomous vehicle has cleared the hazard and when previously operating in the driving-control-system-controlled state.

14. The method of claim 8, further comprising detecting, via the brain-function processor, a gamma power in a visual cortex part of the brain as the parameter of the brain of the driver.

15. A non-transitory, computer-readable media having computer-readable instructions stored thereon, the computer-readable instructions being capable of being read by a vehicle control system for use with a semi-autonomous vehicle, wherein the computer-readable instructions are capable of instructing the vehicle control system to perform the method comprising:
detecting, via hazard-detection processor configured to execute hazard-detection instructions stored in a hazard-detection memory, a hazard;
outputting, via the hazard-detection processor, a hazard-detection signal based on the detected hazard;
detecting, via an eye-tracking processor configured to execute eye-tracking instructions stored in an eye-tracking memory, where a driver of the semi-autonomous vehicle is looking;
outputting, via the eye-tracking processor, an eye-tracking signal based on where the driver is looking;
detecting, via a brain-function processor configured to execute brain-function instructions stored in a brain-function memory, a parameter of a brain of the driver;
outputting, via the brain-function processor, a brain-function signal based on the detected parameter;
determining, via a driving-control processor configured to execute driving-control instructions stored in a driving-control memory, whether the driver comprehends the hazard based on the hazard-detection signal, the eye-tracking signal and the brain-function signal; and
operating the driving-control processor in: a driver-controlled state so as to enable the driver to control at least one of steering of the semi-autonomous vehicle, breaking of the semi-autonomous vehicle, and a combination thereof; and a driving-control-system-controlled state so as to enable the driving-control system to control the at least one of steering of the semi-autonomous vehicle, breaking of the semi-autonomous vehicle, and a combination thereof.

16. The non-transitory, computer-readable media of claim 15, wherein the computer-readable instructions are capable of instructing the vehicle control system to perform the method further comprising providing, via a warning system, a warning related to the driving-control system controlling the at least one of steering of the semi-autonomous vehicle, breaking of the semi-autonomous vehicle, and a combination thereof when the driving-control system is operating in the driving-control system state.

17. The non-transitory, computer-readable media of claim 16, wherein the computer-readable instructions are capable of instructing the vehicle control system to perform the method wherein said comprising a warning comprises displaying, via a display, an image related to the driving-control system controlling the at least one of steering of the semi-autonomous vehicle, breaking of the semi-autonomous vehicle, and a combination thereof when the driving-control system is operating in the driving-control system state.

18. The non-transitory, computer-readable media of claim 17, wherein the computer-readable instructions are capable of instructing the vehicle control system to perform the method wherein said displaying the image comprises displaying the image via a head up display.

19. The non-transitory, computer-readable media of claim 16, wherein the computer-readable instructions are capable of instructing the vehicle control system to perform the method wherein said comprising a warning comprises outputting, via a speaker, an audible warning related to the driving-control system controlling the at least one of steering of the semi-autonomous vehicle, breaking of the semi-autonomous vehicle, and a combination thereof when the driving-control system is operating in the driving-control system state.

20. The non-transitory, computer-readable media of claim 15, wherein the computer-readable instructions are capable of instructing the vehicle control system to perform the method further comprising:
determining, via the driving-control processor, whether the semi-autonomous vehicle has cleared the hazard; and
operating, via the driving-control processor, in the driver controlled state if the semi-autonomous vehicle has cleared the hazard and when previously operating in the driving-control-system-controlled state.
